(19) United States
(12) Patent Application Publication
(10) Pub. No.: US 2026/0283983 A1
(43) Pub. Date: Sep. 24, 2026

(54) PHARMACEUTICAL COMPOSITIONS FOR WOUND CARE

(71) Applicant: BIO PROTECTANT TECHNOLOGIES, INC., Dover, DE (US)

(72) Inventors: Joseph Frederick BUELL, Dover, DE (US); Chad Joseph ROY, Dover, DE (US)

(21) Appl. No.: 19/473,138

(22) PCT Filed: Apr. 5, 2024
(86) PCT No.: PCT/US2024/023331

§ 371 Date: Oct. 6, 2025

Related U.S. Application Data
(60) Provisional application No. 63/457,707, filed on Apr. 6, 2023.

(51) Int. Cl. A61K 31/14; A61K 9/00; A61K 9/06; A61K 9/08; A61K 9/16; A61P 17/02

(52) U.S. Cl. CPC A61K 31/14; A61K 9/0014; A61K 9/06; A61K 9/08; A61K 9/1611; A61K 9/1617; A61P 17/02

(57) ABSTRACT

Provided herein are topical compositions comprising and/or wound care product comprising a quaternary ammonium salt. Also provided herein are methods of treating a wound, such as by stimulating wound healing, enhancing wound healing, and/or accelerating wound healing.

### BACKGROUND OF THE DISCLOSURE

The present disclosure generally relates to the treatment, enhancement, stimulation of wound healing, and/or reducing or elimination of wound infection. Example wounds include, but are not limited to, burns, surgical wounds, and trauma wounds.

### SUMMARY OF THE DISCLOSURE

Disclosed herein is a method for stimulating and/or enhancing wound healing in a subject in need thereof, comprising applying a therapeutically effective amount of a topical pharmaceutical composition comprising a quaternary ammonium salt. Disclosed herein is a method for treating at least one wound in a subject in need thereof, comprising applying a therapeutically effective amount of a topical pharmaceutical composition comprising a quaternary ammonium salt. Disclosed herein is a method for accelerating wound healing in a subject in need thereof, comprising applying a therapeutically effective amount of a topical pharmaceutical composition comprising a quaternary ammonium salt.

In some embodiments, the quaternary ammonium salt is C₄-C₂₀ alkyl(ethylbenzyl)dimethylammonium chloride. In some embodiments, the quaternary ammonium salt is C₆-C₂₀ alkyl(ethylbenzyl)dimethylammonium chloride. In some embodiments, the quaternary ammonium salt is C₆-C₁₈ alkyl(ethylbenzyl)dimethylammonium chloride. In some embodiments, the quaternary ammonium salt is C₈-C₁₈ alkyl(ethylbenzyl)dimethylammonium chloride. In some embodiments, the quaternary ammonium salt is C₈-C₁₆ alkyl(ethylbenzyl)dimethylammonium chloride. In some embodiments, the quaternary ammonium salt is C₁₀-C₁₆ alkyl(ethylbenzyl)dimethylammonium chloride. In some embodiments, the quaternary ammonium salt is C₁₀-C₁₄ alkyl(ethylbenzyl)dimethylammonium chloride. In some embodiments, the quaternary ammonium salt is C₁₂-C₁₄ alkyl(ethylbenzyl)dimethylammonium chloride. In some embodiments, the quaternary ammonium salt is C₁₂ alkyl(ethylbenzyl)dimethylammonium chloride. In some embodiments, the quaternary ammonium salt is C₁₄ alkyl(ethylbenzyl)dimethylammonium chloride.

In some embodiments, the quaternary ammonium salt is present in an amount ranging from about 0.0001% to about 40% w/w. In some embodiments, the quaternary ammonium salt is present in an amount ranging from about 0.0001% to about 20% w/w. In some embodiments, the quaternary ammonium salt is present in an amount ranging from about 0.0001% to about 10% w/w. In some embodiments, the quaternary ammonium salt is present in an amount ranging from about 0.0001% to about 5% w/w. In some embodiments, the quaternary ammonium salt is present in an amount ranging from about 0.0001% to about 4% w/w. In some embodiments, the quaternary ammonium salt is present in an amount ranging from about 0.0001% to about 3% w/w. In some embodiments, the quaternary ammonium salt is present in an amount ranging from about 0.0001% to about 2% w/w. In some embodiments, the quaternary ammonium salt is present in an amount ranging from about 0.0001% to about 1% w/w. In some embodiments, the quaternary ammonium salt is present in an amount ranging from about 0.0001% to about 0.1% w/w. In some embodiments, the quaternary ammonium salt is present in an amount ranging from about 0.0001% to about 0.01% w/w. In some embodiments, the quaternary ammonium salt is present in an amount ranging from about 0.0001% to about 0.001% w/w. In some embodiments, the quaternary ammonium salt is present in an amount ranging from about 0.001% to about 1% w/w. In some embodiments, the quaternary ammonium salt is present in an amount ranging from about 0.01% to about 1% w/w. In some embodiments, the quaternary ammonium salt is present in an amount ranging from about 0.1% to about 1% w/w.

In some embodiments, the pharmaceutical composition further comprises pharmaceutically acceptable carrier. In some embodiments, the pharmaceutically acceptable carrier comprises water, saline, one or more organic solvents, or combinations thereof. In some embodiments, the pharmaceutically acceptable carrier comprises one or more gelling agents, preferably one or more gelling polymers. In some embodiments, the pharmaceutically acceptable carrier comprises an ointment base. In some embodiments, the pharmaceutically acceptable carrier comprises particles, preferably microparticles and/or nanoparticles.

In some embodiments, the pharmaceutical composition is in the form of a solution. In some embodiments, the pharmaceutical composition is in the form of a gel, preferably a hydrogel. In some embodiments, the pharmaceutical composition is in the form of an ointment. In some embodiments, the pharmaceutical composition is in the form of a powder. In some embodiments, the pharmaceutical composition is in the form of a film.

In some embodiments, the pharmaceutical composition further comprises at least one pharmaceutically acceptable excipient. In some embodiments, the at least one pharmaceutically acceptable excipient comprises one or more buffering agents. In some embodiments, the at least one pharmaceutically acceptable excipient comprises one or more viscosity agents. In some embodiments, the at least one pharmaceutically acceptable excipient comprises one or more permeation enhancers. In some embodiments, the at least one pharmaceutically acceptable excipient comprises one or more surfactants. In some embodiments, the at least one pharmaceutically acceptable excipient comprises one or more stabilizers. In some embodiments, the at least one pharmaceutically acceptable excipient comprises one or more emulsifiers. In some embodiments, the at least one pharmaceutically acceptable excipient comprises one or more preservatives. In some embodiments, the at least one pharmaceutically acceptable excipient comprises one or more thickening agents.

In some embodiments, the pharmaceutical composition is incorporated into a wound care product. In some embodiments, the pharmaceutical composition is incorporated into a wound care product as a coating. In some embodiments, the pharmaceutical composition is impregnated throughout a wound care product.

In some embodiments, the wound care product is a wound dressing. In some embodiments, the wound care product is a gauze. In some embodiments, the wound care product is a bandage. In some embodiments, the wound care product is a foam. In some embodiments, the wound care product is a cohesive wrap. In some embodiments, the wound care product is a wound pad, plaster or an absorbent lint.

In some embodiments, the wound care product is a wound wash, and optionally wherein the wound wash is a surgical irrigation liquid or operative debridement liquid. In some embodiments, the wound care product is a surgical wash or surgical scrub.

In some embodiments, the wound is an open wound. In some embodiments, the wound is an incision, an abrasion, a laceration, a puncture, or an avulsion. In some embodiments, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof. In some embodiments, the wound is a burn.

In some embodiments, the wound is infected. In some embodiments, the infected wound is infected with methicillin-resistant Staphylococcus aureus (MRSA).

In some embodiments, the subject is a human. In some embodiments, the subject is immuno-compromised.

Disclosed herein in some embodiments is a wound care product comprising a pharmaceutical composition, wherein the pharmaceutical composition comprises a quaternary ammonium salt.

In some embodiments, the quaternary ammonium salt is C₄-C₂₀ alkyl(ethylbenzyl)dimethylammonium chloride. In some embodiments, the quaternary ammonium salt is C₆-C₂₀ alkyl(ethylbenzyl)dimethylammonium chloride. In some embodiments, the quaternary ammonium salt is C₆-C₁₈ alkyl(ethylbenzyl)dimethylammonium chloride. In some embodiments, the quaternary ammonium salt is C₈-C₁₈ alkyl(ethylbenzyl)dimethylammonium chloride. In some embodiments, the quaternary ammonium salt is C₈-C₁₆ alkyl(ethylbenzyl)dimethylammonium chloride. In some embodiments, the quaternary ammonium salt is C₁₀-C₁₆ alkyl(ethylbenzyl)dimethylammonium chloride. In some embodiments, the quaternary ammonium salt is C₁₀-C₁₄ alkyl(ethylbenzyl)dimethylammonium chloride. In some embodiments, the quaternary ammonium salt is C₁₂-C₁₄ alkyl(ethylbenzyl)dimethylammonium chloride. In some embodiments, the quaternary ammonium salt is C₁₂ alkyl(ethylbenzyl)dimethylammonium chloride. In some embodiments, the quaternary ammonium salt is C₁₄ alkyl(ethylbenzyl)dimethylammonium chloride.

In some embodiments, the quaternary ammonium salt is present in an amount ranging from about 0.0001% to about 40% w/w. In some embodiments, the quaternary ammonium salt is present in an amount ranging from about 0.0001% to about 20% w/w. In some embodiments, the quaternary ammonium salt is present in an amount ranging from about 0.0001% to about 10% w/w. In some embodiments, the quaternary ammonium salt is present in an amount ranging from about 0.0001% to about 5% w/w. In some embodiments, the quaternary ammonium salt is present in an amount ranging from about 0.0001% to about 4% w/w. In some embodiments, the quaternary ammonium salt is present in an amount ranging from about 0.0001% to about 3% w/w. In some embodiments, the quaternary ammonium salt is present in an amount ranging from about 0.0001% to about 2% w/w. In some embodiments, the quaternary ammonium salt is present in an amount ranging from about 0.0001% to about 1% w/w. In some embodiments, the quaternary ammonium salt is present in an amount ranging from about 0.0001% to about 0.1% w/w. In some embodiments, the quaternary ammonium salt is present in an amount ranging from about 0.0001% to about 0.01% w/w. In some embodiments, the quaternary ammonium salt is present in an amount ranging from about 0.0001% to about 0.001% w/w. In some embodiments, the quaternary ammonium salt is present in an amount ranging from about 0.001% to about 1% w/w. In some embodiments, the quaternary ammonium salt is present in an amount ranging from about 0.01% to about 1% w/w. In some embodiments, the quaternary ammonium salt is present in an amount ranging from about 0.1% to about 1% w/w.

In some embodiments, the pharmaceutical composition further comprises pharmaceutically acceptable carrier. In some embodiments, the pharmaceutically acceptable carrier comprises water, saline, one or more organic solvents, or combinations thereof. In some embodiments, the pharmaceutically acceptable carrier comprises one or more gelling agents, preferably one or more gelling polymers. In some embodiments, the pharmaceutically acceptable carrier comprises an ointment base. In some embodiments, the pharmaceutically acceptable carrier comprises particles, preferably microparticles and/or nanoparticles.

In some embodiments, the pharmaceutical composition is in the form of a solution. In some embodiments, the pharmaceutical composition is in the form of a gel, preferably a hydrogel. In some embodiments, the pharmaceutical composition is in the form of an ointment. In some embodiments, the pharmaceutical composition is in the form of a powder. In some embodiments, the pharmaceutical composition is in the form of a film.

In some embodiments, the pharmaceutical composition further comprises at least one pharmaceutically acceptable excipient. In some embodiments, the at least one pharmaceutically acceptable excipient comprises one or more buffering agents. In some embodiments, the at least one pharmaceutically acceptable excipient comprises one or more viscosity agents. In some embodiments, the at least one pharmaceutically acceptable excipient comprises one or more permeation enhancers. In some embodiments, the at least one pharmaceutically acceptable excipient comprises one or more surfactants. In some embodiments, the at least one pharmaceutically acceptable excipient comprises one or more stabilizers. In some embodiments, the at least one pharmaceutically acceptable excipient comprises one or more emulsifiers. In some embodiments, the at least one pharmaceutically acceptable excipient comprises one or more preservatives. In some embodiments, the at least one pharmaceutically acceptable excipient comprises one or more thickening agents.

In some embodiments, the pharmaceutical composition is incorporated into the wound care product as a coating. In some embodiments, the pharmaceutical composition is impregnated throughout the wound care product.

In some embodiments, the wound care product is a wound wash, and optionally wherein the wound wash is a surgical irrigation liquid or operative debridement liquid. In some embodiments, the wound care product is a surgical wash or surgical scrub.

In some embodiments, the wound care product is a wound dressing. In some embodiments, the wound care product is a gauze. In some embodiments, the wound care product is a bandage. In some embodiments, the wound care product is a foam. In some embodiments, the wound care product is a cohesive wrap. In some embodiments, the wound care product is a wound pad, plaster, or an absorbent lint.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows the average radiance in the wound bed detected over a two week period.

FIG. 1B shows the box and whisker plots of the wound area over time, beginning on day 4 after the removal of the surgical wound dressing.

FIG. 1C shows plot of the change in weight.

FIG. 2A shows a wound dressing from the Formulation A treated mice (2,000×).

FIG. 2B shows a wound dressing from the vehicle control treated mice (2,000×).

FIG. 3A shows a wound dressing from the Formulation A treated mice (20,000×).

FIG. 3B shows a wound dressing from the vehicle control treated mice (20,000×).

FIG. 4 shows representative images of a single mouse from each treatment group over the course of the first week of infection. Vehicle control mouse are shown in the bottom. Treated mouse are shown on top.

FIG. 5 shows the results from the quantitative biofilm reduction assay (15 min vs. 60 min) for Formulation B, benzalkonium chloride (BAK) and C₁₂-C₁₄ alkyl(ethylbenzyl)dimethylammonium chloride.

FIG. 6A shows the biofilm accumulation after treatment with Formulation B.

FIG. 6B shows the biofilm accumulation after treatment with C₁₂-C₁₄ alkyl(ethylbenzyl)dimethylammonium chloride.

FIG. 6C shows the biofilm accumulation after treatment with BAK

FIG. 7A shows a representative SEM image of tegaderm covering the wounds of a Formulation B treated mouse magnified at 2,000×.

FIG. 7B shows a representative SEM image of tegaderm covering the wounds of a control treated mouse magnified at 2,000×.

FIG. 8A shows bioluminescence images of mice infected with 10⁴ CA-MRSA treated with either Formulation B, Bacitracin, or vehicle.

FIG. 8B shows the radiance plots (mean±SEM) of mouse wound bed over 7d infected with CA-MRSA.

FIG. 8C shows the wound area measurement (mean±SEM) when treated with either Formulation B or vehicle after surgical dressing removal.

FIG. 8D shows the Gross wound categorical scoring (mean±SEM).

FIG. 8E shows no significant difference observed in daily mean group weights.

### DETAILED DESCRIPTION

Provided herein are, for example, compositions and wound dressings for treating, stimulating and/or enhancing wound healing. Also provided herein are, for example, compositions and wound dressings for reducing scarring.

Detailed descriptions of one or more embodiments are provided herein. It is to be understood, however, that the present disclosure is embodied in various forms. Therefore, specific details disclosed herein are not to be interpreted as limiting, but rather as a basis for the claims and as a representative basis for teaching one skilled in the art to employ the present disclosure in any appropriate manner.

Wherever the phrase “for example,” “such as,” “including,” and the like are used herein, the phrase “and without limitation” is understood to follow unless explicitly stated otherwise. Similarly “an example,” “exemplary,” and the like are understood to be non-limiting.

The term “substantially” allows for deviations from the descriptor that don't negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The term “about” is used to indicate that a value includes the standard level of error for the device or method being employed to determine the value. In some embodiments, the level of error is 10%. In some embodiments, the level of error is 9%. In some embodiments, the level of error is 8%. In some embodiments, the level of error is 7%. In some embodiments, the level of error is 6%. In some embodiments, the level of error is 5%.

The terms “comprising,” “including,” “having,” “involving” (and similarly “comprises,” “includes,” “has,” and “involves”), and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

“Wound care product” as used herein refers to article of manufacture with beneficial effect on stimulating wound healing, accelerating wound healing, enhancing wound healing, alleviating wound infection, and/or treating wound infection.

### Pharmaceutical Compositions

Disclosed herein are pharmaceutical compositions comprising a quaternary ammonium salt. In some embodiments, the quaternary ammonium salt is C12-C14-alkyl(ethylbenzyl)dimethylammonium chloride. In some embodiments, the quaternary ammonium salt is C12-alkyl(ethylbenzyl)dimethylammonium chloride. In some embodiments, the quaternary ammonium salt is C14-alkyl(ethylbenzyl)dimethylammonium chloride. In some embodiments, the pharmaceutical composition further comprises ammonium chloride. In some embodiments, the pharmaceutical composition further comprises a chlorite salt. In some embodiments, the pharmaceutical composition comprises a quaternary ammonium salt, ammonium chloride and a chlorite salt. In some embodiments, the pharmaceutical composition comprises a quaternary ammonium salt and ammonium chloride. In some embodiments, the pharmaceutical composition comprises a quaternary ammonium salt and a chlorite salt. In some embodiments, the pharmaceutical composition is free of alcohols. In some embodiments, the pharmaceutical composition further comprises a pharmaceutically acceptable carrier. In some embodiments, the pharmaceutical composition further comprises a pharmaceutically acceptable excipient. In some embodiments, the pharmaceutically acceptable excipient is selected from buffers, viscosity agents, permeation enhancers, surfactants, stabilizers, emulsifiers, preservatives, thickening agents, and any combinations thereof. Exemplary pharmaceutically acceptable excipients of the disclosure include those found in Remington: The Science and Practice of Pharmacy, Twenty Second Ed. (London, UK: Pharmaceutical Press, 2013) incorporated herein by reference for such disclosure.

### Quaternary Ammonium Salt

In some aspects, the pharmaceutical composition comprises a quaternary ammonium salt. In some embodiments, the quaternary ammonium salt is a salt of a quaternary ammonium cation. As used herein “quaternary ammonium cations” also known as quats, refer to positively charged polyatomic ions of the structure NR₄⁺, R being an optionally substituted alkyl group or an optionally substituted aryl group. Unlike the ammonium ion (NH₄⁺) and the primary, secondary, or tertiary ammonium cations, the quaternary ammonium cations are permanently charged, independent of the pH of their solution. In some embodiments, the quaternary ammonium salt is not a polymeric quaternary ammonium salt. In some embodiments, the quaternary ammonium salt comprises a C10 or C16 alkyl chain. In some embodiments, the quaternary ammonium salt comprises a C12 or C14 alkyl chain. In some embodiments, the quaternary ammonium salt is not benzalkonium chloride. In some embodiments, the quaternary ammonium salt is C10-C16-alkyl(ethylbenzyl)dimethylammonium chloride. In some embodiments, the quaternary ammonium salt is C12-C14-alkyl(ethylbenzyl)dimethylammonium chloride. In some embodiments, the quaternary ammonium salt is C12-alkyl(ethylbenzyl)dimethylammonium chloride. In some embodiments, the quaternary ammonium salt is substantially pure C12-alkyl(ethylbenzyl)dimethylammonium chloride that is separated from a mixture of C12-C14-alkyl(ethylbenzyl)dimethylammonium chloride. In some embodiments, the quaternary ammonium salt is C12-alkyl(ethylbenzyl)dimethylammonium chloride that is separated from a mixture of C12-C14-alkyl(ethylbenzyl)dimethylammonium chloride and contains less than about 1%, about 2%, about 3%, about 4%, about 5%, about 6%, about 7%, about 8%, about 9%, or about 10% of C14-alkyl(ethylbenzyl)dimethylammonium chloride. In some embodiments, the quaternary ammonium salt, e.g., C12-alkyl(ethylbenzyl)dimethylammonium chloride, does not have any toxicity.

In some embodiments, the C12-C14-alkyl(ethylbenzyl)dimethylammonium chloride is a mixture of C12-alkyl(ethylbenzyl)dimethylammonium chloride and C14-alkyl(ethylbenzyl)dimethylammonium chloride.

In some embodiments, the pharmaceutical composition is essentially free of alkyl(ethylbenzyl)dimethyl ammonium salt having an alkyl of less than 12 carbons or more than 14 carbons.

In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01 mg/ml to about 10 mg/ml. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount of about 0.01 mg/ml, about 0.05 mg/ml, about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 1.5 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, or about 10 mg/ml.

In some embodiments, the pharmaceutical composition described herein is substantially free of benzalkonium chloride. In some embodiments, the amount of quaternary ammonium salt is lower than the amount of known quaternary ammonium salts, for example benzalkonium chloride, by about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%.

In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.5% w/w. In some embodiments, the quaternary ammonium salt, e.g., C12-C14-alkyl(ethylbenzyl)dimethylammonium chloride or C12-alkyl(ethylbenzyl)dimethylammonium chloride is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, or about 0.5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.01% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.002% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0005% to about 0.002% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0005% to about 0.0012% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount of about 0.001% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount of about 0.0008% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount of about 0.0001%, about 0.0002%, about 0.0003%, about 0.0004%, about 0.0005%, about 0.0006%, about 0.0007%, about 0.0008%, about 0.0009%, about 0.001%, about 0.005%, about 0.01%, about 0.05%, about 0.1%, about 0.15%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1% w/w, about 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, about 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, or about 10% w/w.

In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount between about 10% w/w and about 20% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount between about 10% w/w and about 15% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount between about 15% w/w and about 20% w/w.

In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount of about 10% w/w, about 10.1% w/w, about 10.2% w/w, about 10.3% w/w, about 10.4% w/w, about 10.5% w/w, about 10.6% w/w, about 10.7% w/w, about 10.8% w/w, about 10.9% w/w, about 11% w/w, about 11.1% w/w, about 11.2% w/w, about 11.3% w/w, about 11.4% w/w, about 11.5% w/w, about 11.6% w/w, about 11.7% w/w, about 11.8% w/w, about 11.9% w/w, about 12% w/w, about 12.1% w/w, about 12.2% w/w, about 12.3% w/w, about 12.4% w/w, about 12.5% w/w, about 12.6% w/w, about 12.7% w/w, about 12.8% w/w, about 12.9% w/w, about 13% w/w, about 13.1% w/w, about 13.2% w/w, about 13.3% w/w, about 13.4% w/w, about 13.5% w/w, about 13.6% w/w, about 13.7% w/w, about 13.8% w/w, about 13.9% w/w, about 14% w/w, about 14.1% w/w, about 14.2% w/w, about 14.3% w/w, about 14.4% w/w, about 14.5% w/w, about 14.6% w/w, about 14.7% w/w, about 14.8% w/w, about 14.9% w/w, about 15% w/w, about 15.1% w/w, about 15.2% w/w, about 15.3% w/w, about 15.4% w/w, about 15.5% w/w, about 15.6% w/w, about 15.7% w/w, about 15.8% w/w, about 15.9% w/w, about 16% w/w, about 16.1% w/w, about 16.2% w/w, about 16.3% w/w, about 16.4% w/w, about 16.5% w/w, about 16.6% w/w, about 16.7% w/w, about 16.8% w/w, about 16.9% w/w, about 17% w/w, about 17.1% w/w, about 17.2% w/w, about 17.3% w/w, about 17.4% w/w, about 17.5% w/w, about 17.6% w/w, about 17.7% w/w, about 17.8% w/w, about 17.9% w/w, about 18% w/w, about 18.1% w/w, about 18.2% w/w, about 18.3% w/w, about 18.4% w/w, about 18.5% w/w, about 18.6% w/w, about 18.7% w/w, about 18.8% w/w, about 18.9% w/w, about 19% w/w, about 19.1% w/w, about 19.2% w/w, about 19.3% w/w, about 19.4% w/w, about 19.5% w/w, about 19.6% w/w, about 19.7% w/w, about 19.8% w/w, about 19.9% w/w, or about 20% w/w.

In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.01% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.0001% to about 0.001% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 0.1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 0.01% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 0.1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.1% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 1% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 2% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 3% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 4% to about 5% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.1% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 1% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 2% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 3% to about 4% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.1% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 1% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 2% to about 3% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.1% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 1% to about 2% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.001% to about 1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.01% to about 1% w/w. In some embodiments, the quaternary ammonium salt, e.g., C12-C14-alkyl(ethylbenzyl)dimethylammonium chloride or C12-alkyl(ethylbenzyl)dimethylammonium chloride, is present in the pharmaceutical composition in an amount ranging from about 0.1% to about 1% w/w. In some embodiments, the quaternary ammonium salt destroys phospholipids within the microbial cell wall, prompting autolysis and microbial cell entry for the oxychlorine-based component in the formulation (e.g. sodium chlorite, stabilized chlorine dioxide, or chlorine dioxide).

### Wound Care Product

In some embodiments, the pharmaceutical composition is incorporated into a wound care product. In some embodiments, the pharmaceutical composition is incorporated into a wound care product as a coating. In some embodiments, the pharmaceutical composition is impregnated throughout a wound care product.

In some embodiments, the wound care product is a wound dressing. In some embodiments, the wound care product is a gauze. In some embodiments, the wound care product is a bandage. In some embodiments, the wound care product is a foam. In some embodiments, the wound care product is a cohesive wrap. In some embodiments, the wound care product is a wound pad or plaster. In some embodiments, the wound care product is an absorbent lint.

In some embodiments, the wound care product is a wound wash. In some embodiments, the wound care product is a surgical wash.

Suitable wound care products include a liquid and/or microorganism impermeable wound dressing, the moisture and gas permeabilities of which can be changed during use without removal of the dressing from the patient. The dressing may comprise an exudate absorbent pad to be in liquid conducting contact with the wound, and a plurality of cover sheets on the outer side of the pad that are of predetermined combined permeability to moisture vapor and gas, and thereby control the microenvironment of the wound. The outer cover sheets may be removable from the dressing, imparting to the dressing the permeability of the remaining cover sheets. Thus, the dressing can be used through a plurality of wound healing conditions, providing different microenvironments to the wound during healing, without removal of the entire dressing from the patient. Also, the dressing can be adapted before use for application to a plurality of wound types, minimizing inventory requirements.

An exemplary wound dressing includes an island dressing that is liquid and microorganism impermeable, and comprises: (a) a liquid and gas permeable exudate absorbing pad having a wound facing inner surface that in use is in liquid conducting contact with the wound, and an opposing outer surface; (b) a plurality of liquid and microorganism impermeable, gas and moisture vapor permeable cover sheets disposed parallel to, adjacent to, and covering the outer surface of the pad; (c) means for adhesively bonding the pad and cover sheets to the skin; and (d) means for sequentially removing outer cover sheets from the outer side of the dressing.

The exemplary island wound dressings may take any convenient form, for instance, a dressing that has a fenestrated base layer to minimize the need for complete dressing changes.

The wound dressings may also have a base layer. The use of a base layer is desirable when a large number of dressing changes will be required during wound healing. The base layer may remain on the patient for up to 10 or more days. During this period the pad and cover sheets may be changed without trauma to the wound that could be caused by removal of the entire dressing from the wound peripheral skin.

The base layer may adhesively attach the dressing to the undamaged skin periphery of the wound. The top of base layer may be adhesively bonded together, holding a pad and inner coversheet in base layer fenestration. An outer cover sheet with tab may be adhesively bonded to the outer side of the periphery of fenestration in an attachment layer. A release liner may cover the skin side of the dressing for ease of handling before use. An attachment layer may not be needed if an inner cover sheet is larger in area than a pad and is bonded directly to the fenestration periphery of base layer.

In use, a pad may function to absorb exudate as it forms in the wound, preventing exudate pooling while maintaining the wound moist. The pad may deliver the exudate to the inner cover sheet, which passes the aqueous portion through as moisture vapor, to the outside cover sheets that remain on the dressing, and on to the outside air.

An absorbent pad may contain one or more layers of absorbent material in adequate quantity. Many non-toxic conventional known absorbent materials are readily available. Exemplary pad materials include gauze, non-woven sheet materials, cellulosic pulp, synthetic pulp, cottony rayon, and absorbent sponges. The pad size and shape will vary with the size and shape of the wound. It should be large enough to cover the wound, and preferably is capable of absorbing at least about 2 to about 20 cc/g of pad of exudate. Optionally, the pad can contain known absorbent particles and medicaments, such as antibiotics and wound healing stimulants.

The wound-facing surface of the pad may be treated with a layer of material that renders the pad non-adhering to the wound without significantly interfering with its absorbing capability. Suitable treatments including lining the wound-facing inner side of the pad with a highly liquid-permeable non-stick layer such as a polymeric net, mesh or perforated film. “Delnet” P530 apertured polyethylene mesh (Applied Extrusion Technology, Inc.) is also suitable. Alternatively, the pad can be coated with a hydrogel material or other non-stick material that does not interfere significantly with exudate absorption.

The term “gas” as used herein, refers to oxygen and carbon dioxide. In practice, normally only the oxygen permeability of a cover sheet material is measured, because carbon dioxide permeabilities of cover sheets are much less critical than oxygen permeabilities. From a practical standpoint, if a cover sheet material has adequate oxygen permeability it also has adequate carbon dioxide permeability.

The inner cover sheet is parallel to, adjacent to, and substantially covers the outer side of the pad. The inner cover sheet and pad may be heat or adhesively bonded thereto. If the dressing has a base layer, the pad also is adhesively bonded to the periphery of the base layer fenestration, either directly or by an attachment layer. The cover sheets are the dressing component essential for controlling the wound microenvironment liquid and gas levels. The cover sheets are liquid and microorganism impermeable and permeable to moisture vapor and gas.

The gas and moisture vapor permeabilities of the dressing change, normally increase, as each successive outer cover sheet is removed from the outer side of the dressing. Each cover sheet is selected so that the overall dressing, that is the combination of cover sheets, has the desired permeabilities for the healing phase during which the dressing will be used initially. Then removal of each successive outer cover sheet will change the moisture and gas permeabilities.

For example, it may be desirable to have during initial use, a dressing with moderate oxygen permeability and low moisture vapor permeability, and subsequently higher oxygen and moisture permeabilities. This can be achieved by using a two cover sheet dressing, the inner cover sheet being selected to have the ultimate use high oxygen and moisture vapor permeabilities, and the outer cover sheet being selected to have oxygen and moisture vapor permeabilities that, in conjunction with the inner cover sheet, will give the initially desired levels of moderate oxygen permeability and low moisture vapor permeability. The dressing with the two cover sheets is applied to the dressing. At the start of the second period of use of the dressing, the outer cover sheet is removed from the dressing, leaving only the inner cover sheet, which thereafter dictates the permeabilities of the dressing.

This same dressing may be used in the initial healing of a wound requiring both high moisture vapor and high oxygen permeabilities, by simply peeling off (by a tab) and discarding before use the outer cover sheet, giving a dressing with the desired high inner cover sheet permeabilities. Thus the dressing has the versatility of being used on wounds requiring either of two distinct microenvironments.

Because the oxygen requirement is high throughout the healing of many wounds, some embodiments of the dressings disclosed herein have oxygen permeabilities of at least about 10,000 cc/m²/day/atm measured at 25° C. and 50% relative humidity; and/or MVTRs of at least about 300 g/m²/day at ambient pressure, 25° C. and 50% R.R., and most preferably in the range of at least about 300 g/m²/day to about 5000 g/m²/day. In some embodiment, the oxygen permeability applies throughout the dressing's usage period, preferably where a plurality or only the inner cover sheet is on the dressing.

In some embodiments, the wound dressing is a bandage. In some embodiments, the wound dressings comprise cotton, gauze, fiberglass, or synthetic material.

### Dosage Forms

Disclosed herein are dosage forms that are applied topically. In some embodiments, the pharmaceutical composition is applied topically to a wound. In some embodiments, the wound is an open wound. In some embodiments, the wound comprises an incision, an abrasion, a laceration, a puncture, or an avulsion. In some embodiments, the pharmaceutical composition is applied directly to the wound or the skin associated with the wound.

In some aspects, the pharmaceutical composition is in the form of a solution, a gel, or an ointment, or any combinations thereof. Exemplary dosage forms of the disclosure include those found in Remington: The Science and Practice of Pharmacy, Twenty Second Ed. (London, UK: Pharmaceutical Press, 2013) incorporated herein by reference for such disclosure.

In some embodiments, the pharmaceutical composition in the form of a solution, a gel, or an ointment, or any combinations thereof, further comprises pharmaceutically acceptable excipients including, viscosity agents, gelling agents, preservatives, oils, penetration enhancers, surfactants, stabilizers, moisturizers, tonicity agents, water, and any combinations thereof.

Penetration enhancers include vitamin E TPGS (Eastman Chemical Company, Kingsport, Tenn.), and other vitamin E derivatives as described in U.S. Pat. No. 6,193,985; and glyceryl monocaprylate/caprate (Cornwell et al. 1998, Int. J. Pharmaceutics, 171 (2): 243-255). In some embodiments, additional penetration enhancers are described in Smith and Maibach (eds.), Percutaneous Penetration Enhancers, CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, I. L. (1997).

In some embodiments, the oils and/or waxy compounds in the pharmaceutical composition are those traditionally employed in the dermatological arts. In some embodiments, the optional oils and/or waxy compounds constitute from 0.5% to 99.9% of the total weight of the composition. The amount of oil and/or wax depends on the actual form or physical state of the composition. Exemplary of such oils are mineral oils (petrolatum); vegetable oils (sweet almond, macadamia, blackcurrant-pip oil); synthetic oils such as perhydrosqualene, fatty alcohols, acids or esters (octyl palmitate, isopropyl lanolate, triglycerides including those of capric/caprylic acids), oxyethylenated or oxypropylenated fatty esters and ethers; and silicone oils (cyclomethicone, polydimethylsiloxanes or PDMS) or fluorinated oils. Exemplary waxy compounds include jojoba oil, paraffin, carnauba wax and beeswax.

Exemplary surfactants (emulsifying and coemulsifying) present in the pharmaceutical composition include the esters of fatty acids and polyethylene glycol (PEG), esters of fatty acids and glycerol (glyceryl stearate) or esters of fatty acids and sugar (sorbitan stearate), as well as the polyoxyethylenated or polyoxypropylenated derivatives thereof, cyclomethicones and dimethicone copolyols, and also anionic surfactants (K or Na alkyl phosphate). In some embodiments, the surfactant is a cationic surfactant. In some embodiments, the surfactant is a zwitterionic surfactant. In some embodiments, the surfactant is a nonionic surfactant. In some embodiments, the surfactant is selected from sodium lauryl sulfate, docusate sodium, polyoxyalkyl ethers, polyoxylalkyl phenyl ethers, polyoxyl castor oils, polyoxyl hydrogenated castor oils, polyoxyl 40 stearates, polyoxy sorbitan esters, sorbitan esters, polysorbates, sorbitan monolaureates, poloxamines, poloxamers, sucrose stearate, sucrose distearate, and any combinations thereof. In some embodiments, the surfactant is a polyoxyethylene-polyoxypropylene block copolymer. In some embodiments, the surfactant is a poloxamer. In some embodiments, the surfactant is a poloxamine. In some embodiments, the surfactant is Tetronic® 908. The surfactant, when used, is typically present in an amount from about 0.01 to 5 weight percent of the composition.

Exemplary stabilizer includes glycol stearate or PEG-150 distearate. The stabilizer, when used, is typically present in an amount from about 0.1 to 5 weight percent of the composition.

Exemplary moisturizers include wheat protein (e.g., laurdimonium hydroxypropyl hydrolyzed wheat protein), hair keratin amino acids, sodium peroxylinecarbolic acid, panthenol, tocopherol (Vitamin E), dimethicone, and the like, and mixtures thereof. Sodium chloride may also be present, particularly when hair keratin amino acids are included as a moisturizer. Moisturizers, when used, are typically present in an amount from about 0.01 to 2 weight percent of the pharmaceutical composition.

Exemplary preservatives include benzyl alcohol, benzalkonium chloride, tetrasodium ethylene-diamine tetraacetic acid (EDTA), methylparaben, propylparaben, benzophenone-4, methylchloroisothiazolinone, methylisothiazolinone, benzethonium chloride, chlorobutanol, phenylmercuric acetate, phenylmercuric nitrate, thimerosal, and any combinations thereof. In some embodiments, the preservative used does not cause patient sensitivity or is not incompatible with the other ingredients in the pharmaceutical composition. Preservatives, when used, are typically present in an amount from about 0.01% to about 10% by weight of the pharmaceutical composition.

Exemplary tonicity agents include sodium chloride, potassium chloride, propylene glycol, dextrose, glycerin, and mannitol.

In some embodiments, the pharmaceutical composition further comprises benzyl alcohol, mineral oil, propylene glycol, sucrose stearate, and sucrose distearate.

In some embodiments, the pharmaceutical composition further comprises cyclomethicone, polyethylene glycol 600, dimethicone, silica, petrolatum, phenyl trimethicone, PEG/PPG-19/19 dimethicone, mica, PEG 12 dimethicone, titanium dioxide, polyurethane-40, menthol, and tin oxide.

### Topical Gel Antibacterial Composition

In some embodiments, the pharmaceutical composition described herein is formulated as a gel. Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a non-limiting example of a hydrophobic gel includes liquid paraffin with polyethylene or fatty oils gelled with colloidal silica, or aluminum or zinc soaps. The base of a non-limiting example of a hydrophilic gel includes water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the pharmaceutical composition disclosed herein is a gel and comprises water and at least one viscosity-enhancing agent. In some embodiment the gel composition described herein is a semi-solid or is in a gelled state before it is topically administered (e.g. at room temperature). In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, and any combinations thereof. In some embodiments, suitable viscosity-enhancing agents include by way of example only, gelling agents and suspending agents. In some embodiments, the enhanced viscosity formulation includes a pharmaceutically acceptable buffer. Sodium chloride or other tonicity agents are optionally used to adjust tonicity, if necessary.

By way of example only, the viscosity-enhancing agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity-enhancing agents include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methylcellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In some embodiments, the viscosity-enhancing excipient is a combination of MCC and CMC. In another embodiment, the viscosity-enhancing agent is a combination of carboxymethylated chitosan, chitin, and alginate. In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiment, the viscosity-enhancing agent is hydroxypropyl methylcellulose.

In one embodiment, the pharmaceutically acceptable enhanced viscosity acceptable formulation comprises at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In some other embodiments, methylcellulose is utilized as the gelling agent. In certain embodiments, the viscosity enhancing agents described herein are also utilized as the gelling agent for the gel formulations presented herein.

In some embodiments, other gel formulations are useful depending upon the pharmaceutical agents or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the pharmaceutical compositions described herein. In some embodiments, acceptable gels include, but are not limited to, alginate hydrogels SAFR-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in the pharmaceutical compositions described herein.

In certain embodiments, the enhanced viscosity formulation is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C., or about 40° C. Included within the definition of body temperature is the body temperature of a healthy individual, or an unhealthy individual, including an individual with a fever (up to ~42° C.). In some embodiments, the pharmaceutical compositions described herein are liquids at about room temperature and are administered at or about room temperature.

Copolymers polyoxypropylene and polyoxyethylene (e.g. polyoxyethylene-polyoxypropylene triblock copolymers) form thermosetting gels when incorporated into aqueous solutions. These polymers have the ability to change from the liquid state to the gel state at temperatures close to body temperature, therefore allowing useful formulations that are applied to the targeted site. The liquid state-to-gel state phase transition is dependent on the polymer concentration and the ingredients in the solution.

In some embodiments, the amount of thermosetting polymer in any formulation described herein is about 10%, about 15%, about 20%, about 25%, about 30%, about 35% or about 40% of the total weight of the formulation. In some embodiments, the amount of thermosetting polymer in any formulation described herein is about 10%, about 11%, about 12%, about 13%, about 14%, about 15%, about 16%, about 17%, about 18%, about 19%, about 20%, about 21%, about 22%, about 23%, about 24% or about 25% of the total weight of the formulation. In some embodiments, the amount of thermosetting polymer (e.g., Poloxamer 407) in any formulation described herein is about 7.5% of the total weight of the formulation. In some embodiments, the amount of thermosetting polymer (e.g., Poloxamer 407) in any formulation described herein is about 10% of the total weight of the formulation. In some embodiments, the amount of thermosetting polymer (e.g., Poloxamer 407) in any formulation described herein is about 11% of the total weight of the formulation. In some embodiments, the amount of thermosetting polymer (e.g., Poloxamer 407) in any formulation described herein is about 12% of the total weight of the formulation. In some embodiments, the amount of thermosetting polymer (e.g., Poloxamer 407) in any formulation described herein is about 13% of the total weight of the formulation. In some embodiments, the amount of thermosetting polymer (e.g., Poloxamer 407) in any formulation described herein is about 14% of the total weight of the formulation. In some embodiments, the amount of thermosetting polymer (e.g., Poloxamer 407) in any formulation described herein is about 15% of the total weight of the formulation. In some embodiments, the amount of thermosetting polymer (e.g., Poloxamer 407) in any formulation described herein is about 16% of the total weight of the formulation. In some embodiments, the amount of thermosetting polymer (e.g., Poloxamer 407) in any formulation described herein is about 17% of the total weight of the formulation. In some embodiments, the amount of thermosetting polymer (e.g., Poloxamer 407) in any formulation described herein is about 18% of the total weight of the formulation. In some embodiments, the amount of thermosetting polymer (e.g., Poloxamer 407) in any formulation described herein is about 19% of the total weight of the formulation. In some embodiments, the amount of thermosetting polymer (e.g., Poloxamer 407) in any formulation described herein is about 20% of the total weight of the formulation. In some embodiments, the amount of thermosetting polymer (e.g., Poloxamer 407) in any formulation described herein is about 21% of the total weight of the formulation. In some embodiments, the amount of thermosetting polymer (e.g., Poloxamer 407) in any formulation described herein is about 23% of the total weight of the formulation. In some embodiments, the amount of thermosetting polymer (e.g., Poloxamer 407) in any formulation described herein is about 25% of the total weight of the formulation. In some embodiments, the amount of thickening agent (e.g., a gelling agent) in any formulation described herein is about 1%, about 5%, about 10%, or about 15% of the total weight of the formulation. In some embodiments, the amount of thickening agent (e.g., a gelling agent) in any formulation described herein is about 0.5%, about 1%, about 1.5%, about 2%, about 2.5%, about 3%, about 3.5%, about 4%, about 4.5%, or about 5% of the total weight of the formulation.

In an alternative embodiment, the thermogel is a PEG-PLGA-PEG triblock copolymer (Jeong etal, Nature (1997), 388:860-2; Jeong etal, J. Control. Release (2000), 63:155-63; Jeong etal, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting coploymers are soluble in water and form a free-flowing liquid at room temperature, but form a hydrogel at body temperature. A commercially available PEG-PLGA-PEG triblock copolymer is RESOMER RGP t50106 manufactured by Boehringer Ingelheim. This material is composed of a PLGA copolymer of 50:50 poly(DL-lactide-co-glycolide) and is 10% w/w of PEG and has a molecular weight of about 6000.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermosetting gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermosetting gel polymer. The active agents and various additives such as buffers, salts, and preservatives are subsequently added and dissolved. In some instances the pharmaceutically agent is suspended if it is insoluble in water. The pH is modulated by the addition of appropriate buffering agents. Gels have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

In some embodiments, gels are also classified as being hydrophobic or hydrophilic. In certain embodiments, the base of a non-limiting example of a hydrophobic gel includes a liquid paraffin with polyethylene or fatty oils gelled with colloidal silica, or aluminum or zinc soaps. In contrast, the base of a non-limiting example of a hydrophilic gel includes water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions disclosed herein is pseudo plastic, plastic, thixotropic, or dilatant.

In some embodiments, the topical composition is an topical gel, and wherein the topically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

In some embodiment, the topical gel composition described herein is a semi-solid or id in a gelled state before it is topically administered (e.g. at room temperature). For example, suitable viscosity-enhancing agents for such gels include by way of example only, gelling agents and suspending agents. In one embodiment, the enhanced viscosity formulation does not include a buffer. In other embodiments, the enhanced viscosity formulation includes a pharmaceutically acceptable buffer. Sodium chloride or other tonicity agents are optionally used to adjust tonicity, if necessary.

By way of example only, the topically acceptable viscosity agent includes hydroxypropyl methylcellulose, hydroxyethyl cellulose, polyvinylpyrrolidone, carboxymethyl cellulose, polyvinyl alcohol, sodium chondroitin sulfate, sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose) or combinations thereof. In specific embodiments, the viscosity-enhancing excipient is a combination of MCC and CMC. In another embodiment, the viscosity-enhancing agent is a combination of carboxymethylated chitosan, or chitin, and alginate. The combination of chitin and alginate with the topical agents disclosed herein acts as a controlled release formulation, restricting the diffusion of the topical agents from the formulation. Moreover, the combination of carboxymethylated chitosan and alginate is optionally used to assist in increasing the permeability of the topical agents in the eye.

In some embodiments is an enhanced viscosity formulation, comprising from about 0.1 mM and about 100 mM of an topical agent, a pharmaceutically acceptable viscosity agent, and water for injection, the concentration of the viscosity agent in the water being sufficient to provide an enhanced viscosity formulation with a final viscosity from about 100 to about 100,000 cP. In certain embodiments, the viscosity of the gel is in the range from about 100 to about 50,000 cP, about 100 cP to about 1,000 cP, about 500 cP to about 1500 cP, about 1000 cP to about 3000 cP, about 2000 cP to about 8,000 cP, about 4,000 cP to about 50,000 cP, about 10,000 cP to about 500,000 cP, about 15,000 cP to about 1,000,000 cP. In other embodiments, when an even more viscous medium is desired, the biocompatible gel comprises at least about 35%, at least about 45%, at least about 55%, at least about 65%, at least about 70%, at least about 75%, or even at least about 80% or so by weight of the topical agent. In highly concentrated samples, the biocompatible enhanced viscosity formulation comprises at least about 25%, at least about 35%, at least about 45%, at least about 55%, at least about 65%, at least about 75%, at least about 85%, at least about 90% or at least about 95% or more by weight of the topical agent.

In one embodiment, the pharmaceutically acceptable enhanced viscosity topically acceptable formulation comprises at least one topical agent and at least one gelling agent. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. In some other embodiments, hydroxypropylmethylcellulose (Methocel®) is utilized as the gelling agent. In certain embodiments, the viscosity enhancing agents described herein are also utilized as the gelling agent for the gel formulations presented herein.

In some embodiments, the topical gel composition described herein is an in situ gel formulation. In some instances, the in situ gel formation is based on increased pre-corneal residence time of the topical composition which improves ocular bioavailability, corneal mucoadhesion, lysosomal interaction and ionic gelation, improved corneal absorption, thermal gelation, or a combination thereof. In some instances, the in situ gel formulation is activated by pH, temperature, ion, UV, or solvent exchange.

In some instances, the topical gel composition comprises a Antibacterial and one or more gelling agents. In some instances, the gelling agent includes, but is not limited to, poloxamer (e.g. Poloxamer 407), tetronics, ethyl (hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g. Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrilic acid (PMMA), polyethylene glycol (PEG), pseudolatexes, xyloglucans, or combinations thereof.

In some instances, the in situ gel formation further comprises a permeation enhancer. In some instances, the permeation enhancer includes surfactants (e.g. non-ionic surfactants), benzalkonium chloride, EDTA, surface-active heteroglycosides, calcium chelators, hydroxyl propyl beta cyclodextrin (HP beta CD), bile salts, and the like.

In some embodiments, other gel formulations are useful depending upon the particular topical agent, other pharmaceutical agent or excipients/additives used, and as such are considered to fall within the scope of the present disclosure. For example, other commercially-available glycerin-based gels, glycerin-derived compounds, conjugated, or crosslinked gels, matrices, hydrogels, and polymers, as well as gelatins and their derivatives, alginates, and alginate-based gels, and even various native and synthetic hydrogel and hydrogel-derived compounds are all expected to be useful in the topical agent formulations described herein. In some embodiments, topically acceptable gels include, but are not limited to, alginate hydrogels SAF®-Gel (ConvaTec, Princeton, N.J.), Duoderm® Hydroactive Gel (ConvaTec), Nu-gel® (Johnson & Johnson Medical, Arlington, Tex.); Carrasyn® (V) Acemannan Hydrogel (Carrington Laboratories, Inc., Irving, Tex.); glycerin gels Elta® Hydrogel (Swiss-American Products, Inc., Dallas, Tex.) and K-Y® Sterile (Johnson & Johnson). In further embodiments, biodegradable biocompatible gels also represent compounds present in topically acceptable formulations disclosed and described herein.

In some embodiments, the viscosity-enhancing agent is a cellulose-based polymer selected from cellulose gum, alkylcellulose, hydroxyl-alkyl cellulose, hydroxyl-alkyl alkylcellulose, carboxy-alkyl cellulose, or combinations thereof. In some embodiments, the viscosity-enhancing agent is hydroxyl-alkyl alkylcellulose. In some embodiment, the viscosity-enhancing agent is hydroxypropyl methylcellulose.

In certain embodiments, the enhanced viscosity formulation is characterized by a phase transition between room temperature and body temperature (including an individual with a serious fever, e.g., up to about 42° C.). In some embodiments, the phase transition occurs at 1° C. below body temperature, at 2° C. below body temperature, at 3° C. below body temperature, at 4° C. below body temperature, at 6° C. below body temperature, at 8° C. below body temperature, or at 10° C. below body temperature. In some embodiments, the phase transition occurs at about 15° C. below body temperature, at about 20° C. below body temperature or at about 25° C. below body temperature. In specific embodiments, the gelation temperature (Tgel) of a formulation described herein is about 20° C., about 25° C., or about 30° C. In certain embodiments, the gelation temperature (Tgel) of a formulation described herein is about 35° C., or about 40° C. Included within the definition of body temperature is the body temperature of a healthy individual, or an unhealthy individual, including an individual with a fever (up to ~42° C.). In some embodiments, the pharmaceutical compositions described herein are liquids at about room temperature and are administered at or about room temperature.

Copolymers polyoxypropylene and polyoxyethylene (e.g. polyoxyethylene-polyoxypropylene triblock copolymers) form thermosetting gels when incorporated into aqueous solutions. These polymers have the ability to change from the liquid state to the gel state at temperatures close to body temperature, therefore allowing useful formulations that are applied to the targeted ocular site. The liquid state-to-gel state phase transition is dependent on the polymer concentration and the ingredients in the solution.

In some embodiments, the amount of thermosetting polymer in any formulation described herein is about 10%, about 15%, about 20%, about 25%, about 30%, about 35% or about 40% of the total weight of the formulation. In some embodiments, the amount of thermosetting polymer in any formulation described herein is about 10%, about 11%, about 12%, about 13%, about 14%, about 15%, about 16%, about 17%, about 18%, about 19%, about 20%, about 21%, about 22%, about 23%, about 24% or about 25% of the total weight of the formulation. In some embodiments, the amount of thermosetting polymer (e.g., Poloxamer 407) in any formulation described herein is about 7.5% of the total weight of the formulation. In some embodiments, the amount of thermosetting polymer (e.g., Poloxamer 407) in any formulation described herein is about 10% of the total weight of the formulation. In some embodiments, the amount of thermosetting polymer (e.g., Poloxamer 407) in any formulation described herein is about 11% of the total weight of the formulation. In some embodiments, the amount of thermosetting polymer (e.g., Poloxamer 407) in any formulation described herein is about 12% of the total weight of the formulation. In some embodiments, the amount of thermosetting polymer (e.g., Poloxamer 407) in any formulation described herein is about 13% of the total weight of the formulation. In some embodiments, the amount of thermosetting polymer (e.g., Poloxamer 407) in any formulation described herein is about 14% of the total weight of the formulation. In some embodiments, the amount of thermosetting polymer (e.g., Poloxamer 407) in any formulation described herein is about 15% of the total weight of the formulation. In some embodiments, the amount of thermosetting polymer (e.g., Poloxamer 407) in any formulation described herein is about 16% of the total weight of the formulation. In some embodiments, the amount of thermosetting polymer (e.g., Poloxamer 407) in any formulation described herein is about 17% of the total weight of the formulation. In some embodiments, the amount of thermosetting polymer (e.g., Poloxamer 407) in any formulation described herein is about 18% of the total weight of the formulation. In some embodiments, the amount of thermosetting polymer (e.g., Poloxamer 407) in any formulation described herein is about 19% of the total weight of the formulation. In some embodiments, the amount of thermosetting polymer (e.g., Poloxamer 407) in any formulation described herein is about 20% of the total weight of the formulation. In some embodiments, the amount of thermosetting polymer (e.g., Poloxamer 407) in any formulation described herein is about 21% of the total weight of the formulation. In some embodiments, the amount of thermosetting polymer (e.g., Poloxamer 407) in any formulation described herein is about 23% of the total weight of the formulation. In some embodiments, the amount of thermosetting polymer (e.g., Poloxamer 407) in any formulation described herein is about 25% of the total weight of the formulation. In some embodiments, the amount of thickening agent (e.g., a gelling agent) in any formulation described herein is about 1%, about 5%, about 10%, or about 15% of the total weight of the formulation. In some embodiments, the amount of thickening agent (e.g., a gelling agent) in any formulation described herein is about 0.5%, about 1%, about 1.5%, about 2%, about 2.5%, about 3%, about 3.5%, about 4%, about 4.5%, or about 5% of the total weight of the formulation.

In an alternative embodiment, the thermogel is a PEG-PLGA-PEG triblock copolymer (Jeong etal, Nature (1997), 388:860-2; Jeong etal, J. Control. Release (2000), 63:155-63; Jeong etal, Adv. Drug Delivery Rev. (2002), 54:37-51). The polymer exhibits sol-gel behavior over a concentration of about 5% w/w to about 40% w/w. Depending on the properties desired, the lactide/glycolide molar ratio in the PLGA copolymer ranges from about 1:1 to about 20:1. The resulting copolymers are soluble in water and form a free-flowing liquid at room temperature, but form a hydrogel at body temperature. A commercially available PEG-PLGA-PEG triblock copolymer is RESOMER RGP t50106 manufactured by Boehringer Ingelheim. This material is composed of a PLGA copolymer of 50:50 poly(DL-lactide-co-glycolide) and is 10% w/w of PEG and has a molecular weight of about 6000.

Additional biodegradable thermoplastic polyesters include AtriGel® (provided by Atrix Laboratories, Inc.) and/or those disclosed, e.g., in U.S. Pat. Nos. 5,324,519; 4,938,763; 5,702,716; 5,744,153; and 5,990,194; wherein the suitable biodegradable thermoplastic polyester is disclosed as a thermoplastic polymer. Examples of suitable biodegradable thermoplastic polyesters include polylactides, polyglycolides, polycaprolactones, copolymers thereof, terpolymers thereof, and any combinations thereof. In some such embodiments, the suitable biodegradable thermoplastic polyester is a polylactide, a polyglycolide, a copolymer thereof, a terpolymer thereof, or a combination thereof. In one embodiment, the biodegradable thermoplastic polyester is 50/50 poly(DL-lactide-co-glycolide) having a carboxy terminal group; is present in about 30 wt. % to about 40 wt. % of the composition; and has an average molecular weight of about 23,000 to about 45,000. Alternatively, in another embodiment, the biodegradable thermoplastic polyester is 75/25 poly (DL-lactide-co-glycolide) without a carboxy terminal group; is present in about 40 wt. % to about 50 wt. % of the composition; and has an average molecular weight of about 15,000 to about 24,000. In further or alternative embodiments, the terminal groups of the poly(DL-lactide-co-glycolide) are either hydroxyl, carboxyl, or ester depending upon the method of polymerization. Polycondensation of lactic or glycolic acid provides a polymer with terminal hydroxyl and carboxyl groups. Ring-opening polymerization of the cyclic lactide or glycolide monomers with water, lactic acid, or glycolic acid provides polymers with the same terminal groups. However, ring-opening of the cyclic monomers with a monofunctional alcohol such as methanol, ethanol, or 1-dodecanol provides a polymer with one hydroxyl group and one ester terminal groups. Ring-opening polymerization of the cyclic monomers with a diol such as 1,6-hexanediol or polyethylene glycol provides a polymer with only hydroxyl terminal groups.

Since the polymer systems of thermosetting gels dissolve more completely at reduced temperatures, methods of solubilization include adding the required amount of polymer to the amount of water to be used at reduced temperatures. Generally after wetting the polymer by shaking, the mixture is capped and placed in a cold chamber or in a thermostatic container at about 0-10° C. in order to dissolve the polymer. The mixture is stirred or shaken to bring about a more rapid dissolution of the thermosetting gel polymer. The topical agent and various additives such as buffers, salts, and preservatives are subsequently added and dissolved. In some instances the pharmaceutically agent is suspended if it is insoluble in water. The pD is modulated by the addition of appropriate buffering agents.

### Topical Ointment Antibacterial Composition

An ointment is a homogeneous, viscous, semi-solid preparation, most commonly a greasy, thick oil (e.g. oil 80%-water 20%) with a high viscosity, intended for external application to the skin or mucous membranes. Ointments have a water number that defines the maximum amount of water that it contains. They are used as emollients or for the application of active ingredients to the skin for protective, therapeutic, or prophylactic purposes and where a degree of occlusion is desired. Ointments are used topically on a variety of body surfaces. These include the skin and the mucous membranes of the eye (an eye ointment), vulva, anus, and nose

The vehicle of an ointment is known as the ointment base. The choice of a base depends upon the clinical indication for the ointment. The different types of ointment bases are: hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil.

Ointments are formulated using hydrophobic, hydrophilic, or water-emulsifying bases to provide preparations that are immiscible, miscible, or emulsifiable with skin secretions. In some embodiments, they are also derived from hydrocarbon (fatty), absorption, water-removable, or water-soluble bases. The active agents are dispersed in the base, and later they get divided after the drug penetration into the target sites (e.g. membranes, skins, etc.).

The present disclosure recognizes that it is sometimes difficult to incorporate into the ointment a drug of low concentration with sufficient dose-to-dose uniformity for effectively treating a disorder or disease. In some embodiments, poly(ethylene-glycols), polyethoxylated castor oils (CremophorREL), alcohols having 12 to 20 carbon atoms or a mixture of two or more of said components are effective excipients for dispersing and/or dissolving effective amounts of topical drugs, in particular of ascomycins and staurosporine derivatives, in an ointment base, in particular in an ointment base substantially comprising oleaginous and hydrocarbon components, and that the resulting ointments are excellently tolerated by the skin and by ocular tissue.

The present disclosure further recognizes that topical drugs, such as a Antibacterial (e.g. atropine or its pharmaceutically acceptable salts), incorporated in the ointment compositions describes herein target the choroid and/or retina in a patient when the compositions are topically administered to the ocular surface, in particular to the sclera of said patient. In some embodiments, an topical ointment composition includes an topical drug, an ointment base and an agent for dispersing and/or dissolving said drug in the ointment base, selected from a poly(ethylene-glycol), a polyethoxylated castor oil, an alcohol having 12 to 20 carbon atoms and a mixture of two or more of said components.

In some embodiments, the ointment bases include topically acceptable oil and fat bases, such as natural wax e.g. white and yellow bees wax, carnauba wax, wool wax (wool fat), purified lanolin, anhydrous lanolin; petroleum wax e.g. hard paraffin, microcrystalline wax; hydrocarbons e.g. liquid paraffin, white and yellow soft paraffin, white petrolatum, yellow petrolatum; or combinations thereof.

The above mentioned oil and fat bases are described in more detail, for instance, in the British Pharmacopoeia, Edition 2001, or the European Pharmacopoeia, 3rd Edition.

In some embodiments, the ointment base is present in amounts of about 50 to about 95, preferably of 70 to 90% by weight based on the total weight of the composition.

A preferred ointment base comprises a combination of one or more of one or more natural waxes like those indicated above, preferably wool wax (wool fat), and one or more hydrocarbons like those indicated above, preferably a soft paraffin or a petrolatum, more preferably in combination with liquid paraffin.

A special embodiment of the aforementioned ointment base comprises e.g. 5 to 17 parts by weight of wool fat, and 50 to 65 parts by weight of white petrolatum as well as 20 to 30 parts by weight of liquid paraffin.

In some embodiments, the agent for dispersing and/or dissolving the topical drug in the ointment base is selected from a poly(ethylene-glycol), a polyethoxylated castor oil, an alcohol having 12 to 20 carbon atoms and a mixture of two or more of said components. The agent is preferably used in amounts of 1 to 20 percent, more preferably 1 to 10 percent by weight of the entire semisolid topical composition.

Alcohols having 12 to 20 carbon atoms include particularly stearyl alcohol (C18H37OH), cetyl alcohol (C16H33OH) and mixtures thereof. Preferred are so-called cetostearyl alcohols, mixtures of solid alcohols substantially consisting of stearyl and cetyl alcohol and preferably comprising not less than 40 percent by weight of stearyl alcohol and a sum of stearyl alcohol and cetyl alcohol amounting to at least 90 percent by weight, and compositions comprising not less than 80 percent by weight of cetylstearyl alcohol and an emulsifier, in particular sodium cetostearyl sulfate and/or sodium lauryl sulfate, preferably in amounts not less than 7 percent by weight of emulsifier.

Polyethoxylated castor oils are reaction products of natural or hydrogenated castor oils and ethylene glycol. In some instances, such products are obtained in known manner, e.g. by reaction of a natural or hydrogenated castor oil or fractions thereof with ethylene oxide, e.g. in a molar ratio of from about 1:30 to about 1:60, with optional removal of free polyethylene glycol components from the product, e.g. in accordance with the methods disclosed in German Auslegeschriften 1,182,388 and 1,518,819. Especially suitable and preferred is a product commercially available under the trade name Cremophor®EL having a molecular weight (by steam osmometry)=ca. 1630, a saponification no.=ca. 65-70, an acid no.=ca. 2, an iodine no.=ca. 28-32 and an nD 25=ca.1.471. Also suitable for use in this category is, for instance, Nikkol®HCO-60, a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0° C.; Freezing point=ca. 32.4° C.; H₂O content (%, KF)=ca. 0.03.

Poly(ethylene-glycols) are used in some embodiments as the agent for dispersing and/or dissolving the topical drug in the ointment base according to the present disclosure. Suitable poly(ethylene-glycol) s are typically mixtures of polymeric compounds of the general formula H—(OCH2-CH2)nOH, wherein the index n typically range from 4 to 230 and the mean molecular weight from about 200 to about 10000. Preferably n is a number from about 6 to about 22 and the mean molecular weight between about 300 and about 1000, more preferably n ranges from about 6 to about 13 and the mean molecular weight from about 300 to about 600, most preferably n has a value of about 8.5 to about 9 and the relative molecular weight is about 400. Suitable poly(ethylene-glycols) are readily available commercially, for example poly(ethylene-glycols) having a mean molecular weight of about 200, 300, 400, 600, 1000, 1500, 2000, 3000, 4000, 6000, 8000 and 10000.

The poly(ethylene-glycols), in particular the preferred types described in the foregoing paragraph, are preferably used in amounts of 1 to 10, more preferably 1 to 5 percent by weight of the entire semisolid topical composition.

An especially preferred embodiment of the compositions according to the instant disclosure comprises an agent for dispersing and/or dissolving of the drug in the ointment base which is selected from a poly(ethylene-glycol), a polyethoxylated castor oil and preferably a mixture of said components.

In some embodiments, the pharmaceutical composition described herein is formulated as an ointment. An ointment is a homogeneous, viscous, semi-solid preparation, most commonly a greasy, thick oil (e.g. oil 80%-water 20%) with a high viscosity, intended for topical application to the skin or mucous membranes. Ointments have a Water number that defines the maximum amount of water that it can contain. They are used as emollients or for the application of active ingredients to the skin for protective, therapeutic, or prophylactic purposes and where a degree of occlusion is desired. Ointments are used topically on a variety of body surfaces. These include the skin and the mucous membranes.

The vehicle of an ointment is known as the ointment base. The choice of a base depends upon the clinical indication for the ointment. The different types of ointment bases are: hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil.

Ointments are formulated using hydrophobic, hydrophilic, or water-emulsifying bases to provide preparations that are immiscible, miscible, or emulsifiable with skin secretions. They can also be derived from hydrocarbon (fatty), absorption, water-removable, or water-soluble bases. The active agents are dispersed in the base, and later they get divided after the drug penetration into the target sites (e.g. skin, etc.).

The present disclosure recognizes that it is sometimes difficult to incorporate into the ointment a drug of low concentration with sufficient dose-to-dose uniformity for effectively treating a disorder or disease. In some embodiments, poly(ethylene-glycols), polyethoxylated castor oils (Cremophor®EL), alcohols having 12 to 20 carbon atoms or a mixture of two or more of said components are effective excipients for dispersing and/or dissolving effective amounts of active agents, in an ointment base, in particular in an ointment base substantially comprising oleaginous and hydrocarbon components, and that the resulting ointments are excellently tolerated by the skin.

In some embodiments, the ointment bases include pharmaceutically acceptable oil and fat bases, such as natural wax e.g. white and yellow bees wax, carnauba wax, wool wax (wool fat), purified lanolin, anhydrous lanolin; petroleum wax e.g. hard paraffin, microcrystalline wax; hydrocarbons e.g. liquid paraffin, white and yellow soft paraffin, white petrolatum, yellow petrolatum; or combinations thereof.

The above mentioned oil and fat bases are described in more detail, for instance, in the British Pharmacopoeia, Edition 2001, or the European Pharmacopoeia, 3rd Edition.

In some embodiments, the ointment base is present in amounts of about 50% to about 95%, preferably of about 70% to about 90% by weight based on the total weight of the composition.

Alcohols having 12 to 20 carbon atoms include particularly stearyl alcohol (C₁₈H₃₇OH), cetyl alcohol (C₁₆H₃₃OH) and mixtures thereof. Preferred are so-called cetostearyl alcohols, mixtures of solid alcohols substantially consisting of stearyl and cetyl alcohol and preferably comprising not less than 40 percent by weight of stearyl alcohol and a sum of stearyl alcohol and cetyl alcohol amounting to at least 90 percent by weight, and compositions comprising not less than 80 percent by weight of cetylstearyl alcohol and an emulsifier, in particular sodium cetostearyl sulfate and/or sodium lauryl sulfate, preferably in amounts not less than 7% by weight of emulsifier.

Polyethoxylated castor oils are reaction products of natural or hydrogenated castor oils and ethylene glycol. Such products may be obtained in known manner, e.g. by reaction of a natural or hydrogenated castor oil or fractions thereof with ethylene oxide, e.g. in a molar ratio of from about 1:30 to about 1:60. Especially suitable and preferred is a product commercially available under the trade name Cremophor®EL having a molecular weight (by steam osmometry)=ca. 1630, a saponification no.=ca. 65-70, an acid no.=ca. 2, an iodine no.=ca. 28-32 and an nD 25=ca. 1.471. Also suitable for use in this category is, for instance, NikkolRHCO-60, a reaction product of hydrogenated castor oil and ethylene oxide exhibiting the following characteristics: acid no.=ca. 0.3; saponification no.=ca. 47.4; hydroxy value=ca. 42.5. pH (5%)=ca. 4.6; Color APHA=ca. 40; m.p.=ca. 36.0 C.; Freezing point=ca. 32.4° C.; H₂O content (%, KF)=ca. 0.03.

Poly(ethylene-glycols) are used in some embodiments as the agent for dispersing and/or dissolving the active agents in the ointment base according to the present disclosure. Suitable poly(ethylene-glycol) s are typically mixtures of polymeric compounds of the general formula H—(OCH₂—CH₂)nOH, wherein the index n may typically range from 4 to 230 and the mean molecular weight from about 200 to about 10000. Preferably n is a number from about 6 to about 22 and the mean molecular weight between about 300 and about 1000, more preferably n ranges from about 6 to about 13 and the mean molecular weight from about 300 to about 600, most preferably n has a value of about 8.5 to about 9 and the relative molecular weight is about 400. Suitable poly(ethylene-glycols) are readily available commercially, for example poly(ethylene-glycols) having a mean molecular weight of about 200, 300, 400, 600, 1000, 1500, 2000, 3000, 4000, 6000, 8000 and 10000.

The poly(ethylene-glycols), in particular the preferred types described in the foregoing paragraph, are preferably used in amounts of 1 to 10, more preferably 1 to 5 percent by weight of the entire semisolid composition.

An especially preferred embodiment of the compositions according to the instant disclosure comprises an agent for dispersing and/or dissolving of the drug in the ointment base which is selected from a poly(ethylene-glycol), a polyethoxylated castor oil and preferably a mixture of said components.

### Gel/Ointment Viscosity

In some embodiments, the composition has a Brookfield RVDV viscosity of from about 10,000 to about 300,000 cps at about 20° C. and sheer rate of 1 s⁻¹. In some embodiments, the composition has a Brookfield RVDV viscosity of from about 15,000 to about 200,000 cps at about 20° C. and sheer rate of 1 s⁻¹. In some embodiments, the composition has a Brookfield RVDV viscosity of from about 50,000 to about 150,000 cps at about 20° C. and sheer rate of 1 s⁻¹. In some embodiments, the composition has a Brookfield RVDV viscosity of from about 70,000 to about 130,000 cps at about 20° C. and sheer rate of 1 s⁻¹. In some embodiments, the composition has a Brookfield RVDV viscosity of from about 90,000 to about 110,000 cps at about 20° C. and sheer rate of 1 s⁻¹.

In some embodiments, the topical gel formulation contains a viscosity enhancing agent sufficient to provide a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise. In some embodiments, the topical gel formulation contains a viscosity enhancing agent sufficient to provide a viscosity of between about 50,0000 and 1,000,000 centipoise.

In some embodiments, the compositions described herein are low viscosity compositions at body temperature. In some embodiments, low viscosity compositions contain from about 1% to about 10% of a viscosity enhancing agent (e.g., gelling components such as polyoxyethylene-polyoxypropylene copolymers). In some embodiments, low viscosity compositions contain from about 2% to about 10% of a viscosity enhancing agent (e.g., gelling components such as polyoxyethylene-polyoxypropylene copolymers). In some embodiments, low viscosity compositions contain from about 5% to about 10% of a viscosity enhancing agent (e.g., gelling components such as polyoxyethylene-polyoxypropylene copolymers). In some embodiments, low viscosity compositions are substantially free of a viscosity enhancing agent (e.g., gelling components such as polyoxyethylene-polyoxypropylene copolymers). In some embodiments, a low viscosity topical agent composition described herein provides an apparent viscosity of from about 100 cP to about 10,000 cP. In some embodiments, a low viscosity topical agent composition described herein provides an apparent viscosity of from about 500 cP to about 10,000 cP. In some embodiments, a low viscosity topical agent composition described herein provides an apparent viscosity of from about 1000 cP to about 10,000 cP.

In some embodiments, the compositions described herein are viscous compositions at body temperature. In some embodiments, viscous compositions contain from about 10% to about 25% of a viscosity enhancing agent (e.g., gelling components such as polyoxyethylene-polyoxypropylene copolymers). In some embodiments, the viscous compositions contain from about 14% to about 22% of a viscosity enhancing agent (e.g., gelling components such as polyoxyethylene-polyoxypropylene copolymers). In some embodiments, the viscous compositions contain from about 15% to about 21% of a viscosity enhancing agent (e.g., gelling components such as polyoxyethylene-polyoxypropylene copolymers). In some embodiments, a viscous topical composition described herein provides an apparent viscosity of from about 100,000 cP to about 1,000,000 cP. In some embodiments, a viscous topical composition described herein provides an apparent viscosity of from about 150,000 cP to about 500,000 cP. In some embodiments, a viscous topical composition described herein provides an apparent viscosity of from about 250,000 cP to about 500,000 cP. In some of such embodiments, a viscous topical composition is a liquid at room temperature and gels at about between room temperature and body temperature (including an individual with a serious fever, e.g., up to about 42° C.). In some embodiments, a viscous topical composition is administered as monotherapy for treatment of an topical disease or condition described herein.

In some embodiments, the viscosity of the gel formulations presented herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

### Viscosity

In some embodiments, the pharmaceutical composition disclosed herein has a Brookfield RVDV viscosity of from about 10,000 to about 300,000 cps at about 20° C. and sheer rate of 1 s⁻¹. In some embodiments, the pharmaceutical composition disclosed herein has a Brookfield RVDV viscosity of from about 15,000 to about 200,000 cps at about 20° C. and sheer rate of 1 s⁻¹. In some embodiments, the pharmaceutical composition disclosed herein has a Brookfield RVDV viscosity of from about 50,000 to about 150,000 cps at about 20° C. and sheer rate of 1 s⁻¹. In some embodiments, the pharmaceutical composition disclosed herein has a Brookfield RVDV viscosity of from about 70,000 to about 130,000 cps at about 20° C. and sheer rate of 1 s⁻¹. In some embodiments, the pharmaceutical composition disclosed herein has a Brookfield RVDV viscosity of from about 90,000 to about 110,000 cps at about 20° C. and sheer rate of 1 s⁻¹.

In some embodiments, the pharmaceutical composition disclosed herein contains a viscosity enhancing agent sufficient to provide a viscosity between about 500 and about 1,000,000 centipoise, between about 750 and about 1,000,000 centipoise; between about 1000 and about 1,000,000 centipoise; between about 1000 and about 400,000 centipoise; between about 2000 and about 100,000 centipoise; between about 3000 and about 50,000 centipoise; between about 4000 and about 25,000 centipoise; between about 5000 and about 20,000 centipoise; or between about 6000 and about 15,000 centipoise. In some embodiments, the pharmaceutical composition disclosed herein contains a viscosity enhancing agent sufficient to provide a viscosity of between about 50,0000 and about 1,000,000 centipoise.

In some embodiments, a viscous composition described herein provides an apparent viscosity of from about 100,000 cP to about 1,000,000 cP. In some embodiments, a viscous composition described herein provides an apparent viscosity from about 150,000 cP to about 500,000 cP. In some embodiments, a viscous composition described herein provides an apparent viscosity from about 250,000 cP to about 500,000 cP.

In some embodiments, the viscosity of the pharmaceutical composition disclosed herein is measured by any means described. For example, in some embodiments, an LVDV-II+CP Cone Plate Viscometer and a Cone Spindle CPE-40 is used to calculate the viscosity of the gel formulation described herein. In other embodiments, a Brookfield (spindle and cup) viscometer is used to calculate the viscosity of the gel formulation described herein. In some embodiments, the viscosity ranges referred to herein are measured at room temperature. In other embodiments, the viscosity ranges referred to herein are measured at body temperature (e.g., at the average body temperature of a healthy human).

### Stability

Disclosed herein are stable pharmaceutical compositions. The pharmaceutical compositions described herein are stable in various storage conditions including refrigerated, ambient, and accelerated conditions.

In some embodiments, stable as used herein refers to a pharmaceutical composition having about 5% w/w or less total impurities at the end of a given storage period. Stability is assessed by HPLC or any other known testing method. In some embodiments, the stable pharmaceutical composition has about 5% w/w, about 4% w/w, about 3% w/w, about 2.5% w/w, about 2% w/w, about 1.5% w/w, about 1% w/w, or about 0.5% w/w total impurities at the end of a given storage period. In other embodiments, the pharmaceutical composition has about 5% w/w total impurities. In yet other embodiments, the pharmaceutical composition has about 4% w/w total impurities. In yet other embodiments, pharmaceutical composition has about 3% w/w total impurities. In yet other embodiments, the pharmaceutical composition has about 2% w/w total impurities. In yet other embodiments, the pharmaceutical composition has about 1% w/w total impurities. In yet other embodiments, the pharmaceutical composition has about 0.9% w/w total impurities. In yet other embodiments, the pharmaceutical composition has about 0.8% w/w total impurities. In yet other embodiments, the pharmaceutical composition has about 0.7% w/w total impurities. In yet other embodiments, the pharmaceutical composition has about 0.6% w/w total impurities. In yet other embodiments, the pharmaceutical composition has about 0.5% w/w total impurities. In yet other embodiments, the pharmaceutical composition has about 0.4% w/w total impurities. In yet other embodiments, the pharmaceutical composition has about 0.3% w/w total impurities. In yet other embodiments, the pharmaceutical composition has about 0.2% w/w total impurities. In yet other embodiments, the pharmaceutical composition has about 0.1% w/w total impurities. In yet other embodiments, the pharmaceutical composition has about 0.09% w/w total impurities. In yet other embodiments, the pharmaceutical composition has about 0.08% w/w total impurities. In yet other embodiments, the pharmaceutical composition has about 0.07% w/w total impurities. In yet other embodiments, the pharmaceutical composition has about 0.06% w/w total impurities. In yet other embodiments, the pharmaceutical composition has about 0.05% w/w total impurities. In yet other embodiments, the pharmaceutical composition has about 0.04% w/w total impurities. In yet other embodiments, the pharmaceutical composition has about 0.03% w/w total impurities. In yet other embodiments, the pharmaceutical composition has about 0.02% w/w total impurities. In yet other embodiments, the pharmaceutical composition has about 0.01% w/w total impurities. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

In some embodiments, stable as used herein refers to a pharmaceutical composition having about 10% or less loss of biocidal activity at the end of a given storage period. Biocidal activity is assessed by known testing method. In some embodiments, the stable pharmaceutical composition has about 10%, about 9.5%, about 9%, about 8.5%, about 8%, about 7.5%, about 7%, about 6.5%, about 6%, about 5.5%, about 5%, about 4.5%, about 4%, about 3.5%, about 3%, about 2.5%, about 2%, about 1.5%, about 1%, or about 0.5% loss of biocidal activity at the end of a given storage period. In some embodiments, the stable pharmaceutical composition has zero loss of biocidal activity at the end of a given storage period. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

In some embodiments, stable as used herein refers to a pharmaceutical composition having no sign of precipitation at the end of a given storage period. Precipitation is assessed by known testing method. In some embodiments, at refrigerated condition, the pharmaceutical compositions described herein are stable for at least 1 month, at least 2 months, at least 3 months, at least 6 months, at least 9 months, at least 12 months, at least 15 months, at least 18 months, at least 24 months, at least 30 months and at least 36 months. In some embodiments, at accelerated conditions, the pharmaceutical compositions described herein are stable for at least 1 month, at least 2 months, at least 3 months, at least 4 months, at least 5 months, at least 6 months, at least 7 months, at least 8 months, at least 9 months, at least 10 months, at least 11 months, or at least 12 months.

Methods for Stimulating and/or Enhancing Wound Healing

Disclosed herein are methods for stimulating and/or enhancing wound healing in a subject in need thereof, comprising applying a therapeutically effective amount of a topical pharmaceutical composition described herein. In some embodiments of a method for stimulating and/or enhancing wound healing, the pharmaceutical composition comprises a quaternary ammonium salt. In some embodiments, the quaternary ammonium salt is an alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments, the topical pharmaceutical composition further comprises ammonium chloride. In some embodiments, the topical pharmaceutical composition further comprises a chlorite salt.

In some embodiments of a method for stimulating and/or enhancing wound healing, the wound is an open wound. In some embodiments stimulating and/or enhancing wound healing, the wound is an incision, an abrasion, a laceration, a puncture, or an avulsion. In some embodiments of a method of stimulating and/or enhancing wound healing, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof.

In some embodiments of a method for stimulating and/or enhancing wound healing, the total wound area is about 2 times to about 10 times smaller in size as compared to a non-treated wound. In some embodiments of a method for stimulating and/or enhancing wound healing, the total wound area is about 2 times smaller in size as compared to a non-treated wound. In some embodiments of a method for stimulating and/or enhancing wound healing, the total wound area is about 3 times smaller in size as compared to a non-treated wound. In some embodiments of a method for stimulating and/or enhancing wound healing, the total wound area is about 4 times smaller in size as compared to a non-treated wound. In some embodiments of a method for stimulating and/or enhancing wound healing, the total wound area is about 5 times smaller in size as compared to a non-treated wound. In some embodiments of a method for stimulating and/or enhancing wound healing, the total wound area is about 6 times smaller in size as compared to a non-treated wound. In some embodiments of a method for stimulating and/or enhancing wound healing, the total wound area is about 7 times smaller in size as compared to a non-treated wound. In some embodiments of a method for stimulating and/or enhancing wound healing, the total wound area is about 8 times smaller in size as compared to a non-treated wound. In some embodiments of a method for stimulating and/or enhancing wound healing, the total wound area is about 9 times smaller in size as compared to a non-treated wound. In some embodiments of a method for stimulating and/or enhancing wound healing, the total wound area is about 10 times smaller in size as compared to a non-treated wound.

In some embodiments of a method for stimulating and/or enhancing wound healing, the wound is an open wound. In some embodiments of a method for stimulating and/or enhancing wound healing, the wound is an incision, an abrasion, a laceration, a puncture, or an avulsion. In some embodiments, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof. In some embodiments of a method for stimulating and/or enhancing wound healing, the wound is a burn. In some embodiments of a method for stimulating and/or enhancing wound healing, the wound is infected. In some embodiments of a method for stimulating and/or enhancing wound healing, the subject is a mammal. In some embodiments of a method for stimulating and/or enhancing wound healing, the subject is a domestic animal. In some embodiments of a method for stimulating and/or enhancing wound healing, the subject is a human. In some embodiments of a method for stimulating and/or enhancing wound healing, the subject is immuno-compromised. In some embodiments of a method for stimulating and/or enhancing wound healing, the subject has cancer, is HIV positive, has AIDS, or is taking an immunosuppressant drug.

### Methods for Accelerating Wound Healing

Also disclosed herein are methods for accelerating wound healing in a subject in need thereof, comprising applying a therapeutically effective amount of a topical pharmaceutical composition described herein. In some embodiments of a method for accelerating wound healing, the pharmaceutical composition comprises a quaternary ammonium salt. In some embodiments, the quaternary ammonium salt is an alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments, the topical pharmaceutical composition further comprises ammonium chloride. In some embodiments, the topical pharmaceutical composition further comprises a chlorite salt.

In some embodiments of a method for accelerating wound healing, the wound closes about 2 times to about 10 times faster as compared to a non-treated wound. In some embodiments of a method for accelerating wound healing, the wound closes about 2 times faster as compared to a non-treated wound. In some embodiments of a method for accelerating wound healing, the wound closes about 3 times faster as compared to a non-treated wound. In some embodiments of a method for accelerating wound healing, the wound closes about 4 times faster as compared to a non-treated wound. In some embodiments of a method for accelerating wound healing, the wound closes about 5 times faster as compared to a non-treated wound. In some embodiments of a method for accelerating wound healing, the wound closes about 6 times faster as compared to a non-treated wound. In some embodiments of a method for accelerating wound healing, the wound closes about 7 times faster as compared to a non-treated wound. In some embodiments of a method for accelerating wound healing, the wound closes about 8 times faster as compared to a non-treated wound. In some embodiments of a method for accelerating wound healing, the wound closes about 9 times faster as compared to a non-treated wound. In some embodiments of a method for accelerating wound healing, the wound closes about 10 times faster as compared to a non-treated wound.

In some embodiments of a method for accelerating wound healing, the wound is an open wound. In some embodiments of a method for accelerating wound healing, the wound is an incision, an abrasion, a laceration, a puncture, or an avulsion. In some embodiments of a method for accelerating wound healing, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof. In some embodiments of a method for accelerating wound healing, the wound is a burn. In some embodiments of a method for accelerating wound healing, the wound is infected. In some embodiments of a method for accelerating wound healing, the subject is a mammal. In some embodiments of a method for accelerating wound healing, the subject is a domestic animal. In some embodiments, the subject is a human. In some embodiments of a method for accelerating wound healing, the subject is immuno-compromised. In some embodiments of a method for accelerating wound healing, the subject has cancer, is HIV positive, has AIDS, or is taking an immunosuppressant drug.

### Method of Treating a Wound

Also disclosed herein are methods for treating at least one wound in a subject in need thereof, comprising applying a therapeutically effective amount of a topical pharmaceutical composition described herein. In some embodiments of treating at least one wound, the pharmaceutical composition comprises a quaternary ammonium salt. In some embodiments, the quaternary ammonium salt is an alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments, the topical pharmaceutical composition further comprises ammonium chloride. In some embodiments, the topical pharmaceutical composition further comprises a chlorite salt.

In some embodiments of a method of treating a wound, the wound is an open wound. In some embodiments of a method of treating a wound, the wound is an incision, an abrasion, a laceration, a puncture, or an avulsion. In some embodiments of a method of treating a wound, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof. In some embodiments of a method of treating a wound, the wound is a burn. In some embodiments of a method of treating a wound, the wound is infected. In some embodiments of a method of treating a wound, the subject is a mammal. In some embodiments of a method of treating a wound, the subject is a domestic animal. In some embodiments, the subject is a human.

In some embodiments of a method of treating a wound, the subject is a human. In some embodiments of a method of treating a wound, the subject is immuno-compromised. In some embodiments of a method of treating a wound, the subject has cancer, is HIV positive, has AIDS, or is taking an immunosuppressant drug.

### Methods for Reducing Scarring

Disclosed herein are methods for reducing scarring in a subject in need thereof, comprising applying a therapeutically effective amount of a topical pharmaceutical composition described herein. In some embodiments of a method for reducing scarring, the pharmaceutical composition comprises a quaternary ammonium salt. In some embodiments, the quaternary ammonium salt is an alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments, the topical pharmaceutical composition further comprises ammonium chloride. In some embodiments, the topical pharmaceutical composition further comprises a chlorite salt.

In some embodiments of a method for reducing scarring, the wound is an open wound. In some embodiments of a method for reducing scarring, the wound is an incision, an abrasion, a laceration, a puncture, or an avulsion. In some embodiments of a method for reducing scarring, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof.

In some embodiments of a method for reducing scarring, the scar is about 2 times to about 10 times smaller in size as compared to a non-treated wound. In some embodiments of a method for reducing scarring, the scar is about 2 times smaller in size as compared to a non-treated wound. In some embodiments of a method for reducing scarring, the scar is about 3 times smaller in size as compared to a non-treated wound. In some embodiments of a method for reducing scarring, the scar is about 4 times smaller in size as compared to a non-treated wound. In some embodiments of a method for reducing scarring, the scar is about 5 times smaller in size as compared to a non-treated wound. In some embodiments of a method for reducing scarring, the scar is about 6 times smaller in size as compared to a non-treated wound. In some embodiments of a method for reducing scarring, the scar is about 7 times smaller in size as compared to a non-treated wound. In some embodiments of a method for reducing scarring, the scar is about 8 times smaller in size as compared to a non-treated wound. In some embodiments of a method for reducing scarring, the scar is about 9 times smaller in size as compared to a non-treated wound. In some embodiments of a method for reducing scarring, the scar is about 10 times smaller in size as compared to a non-treated wound.

In some embodiments of a method for reducing scarring, the wound is an open wound. In some embodiments of a method for reducing scarring, the wound is an incision, an abrasion, a laceration, a puncture, or an avulsion. In some embodiments of a method for reducing scarring, the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof. In some embodiments of a method for reducing scarring, the wound is a burn. In some embodiments of a method for reducing scarring, the wound is infected. In some embodiments of a method for reducing scarring, the subject is a mammal. In some embodiments of a method for reducing scarring, the subject is a domestic animal. In some embodiments, the subject is a human. In some embodiments of a method for reducing scarring, the subject is a human. In some embodiments of a method for reducing scarring, the subject is immuno-compromised. In some embodiments of a method for reducing scarring, the subject has cancer, is HIV positive, has AIDS, or is taking an immunosuppressant drug.

### Method of Treating a Burn

Also disclosed herein are methods of treating a burn in a subject in need thereof, comprising applying a therapeutically effective amount of a topical pharmaceutical composition described herein. In some embodiments of a method of treating a burn, the pharmaceutical composition comprises a quaternary ammonium salt. In some embodiments of a method of treating a burn, the quaternary ammonium salt is an alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments of a method of treating a burn, the topical pharmaceutical composition further comprises ammonium chloride. In some embodiments of a method of treating a burn, the topical pharmaceutical composition further comprises a chlorite salt.

In some embodiments of a method of treating a burn, the burn is infected. In some embodiments of a method of treating a burn, the subject is a mammal. In some embodiments of a method of treating a burn, the subject is a domestic animal. In some embodiments, the subject is a human. In some embodiments of a method of treating a burn, the subject is immuno-compromised. In some embodiments of a method of treating a burn, the subject has cancer, is HIV positive, has AIDS, or is taking an immunosuppressant drug.

### Methods of Reducing the Formation of Biofilm

Also disclosed herein are methods of reducing the formation of biofilm.

A biofilm is any group of microorganisms in which cells stick to each other and often these cells adhere to a surface. These adherent cells are frequently embedded within a self-produced matrix of extracellular polymeric substance (EPS). The biofilm EPS is typically comprised of a polymeric conglomeration generally composed of extracellular DNA, proteins, and polysaccharides. Biofilms may form on living or non-living surfaces and can be prevalent in natural, industrial and hospital settings. The microbial cells growing in a biofilm are physiologically distinct from planktonic cells of the same organism, which, by contrast, are single-cells that may float or swim in a liquid medium.

### Mode of Administration

In some embodiments, the pharmaceutical composition described herein is applied topically to the skin or wound.

In some embodiments, the pharmaceutical composition described herein is applied topically once per day, twice per day, three times per day, four times per day, five times per day or more frequent, every day, once per week, twice per week, three times per week, four times per week, five times per week, six times per week, every other day, every other week, once per month, twice per month, three times per month, continuously over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or more. In some embodiments, the pharmaceutical composition is applied for at least 10 days. In some embodiments, the pharmaceutical composition is applied for at least 9 days. In some embodiments, the pharmaceutical composition is applied for at least 8 days. In some embodiments, the pharmaceutical composition is applied for at least 7 days. In some embodiments, the pharmaceutical composition is applied for at least 6 days. In some embodiments, the pharmaceutical composition is applied for at least 5 days. In some embodiments, the pharmaceutical composition is applied for at least 4 days. In some embodiments, the pharmaceutical composition is applied for at least 3 days.

In some embodiments, the pharmaceutical composition is applied for about 14 days. In some embodiments, the pharmaceutical composition is applied for about 13 days. In some embodiments, the pharmaceutical composition is applied for about 12 days. In some embodiments, the pharmaceutical composition is applied for about 11 days. In some embodiments, the pharmaceutical composition is applied for about 10 days. In some embodiments, the pharmaceutical composition is applied for about 9 days. In some embodiments, the pharmaceutical composition is applied for about 8 days. In some embodiments, the pharmaceutical composition is applied for about 7 days. In some embodiments, the pharmaceutical composition is applied for about 6 days. In some embodiments, the pharmaceutical composition is applied for about 5 days. In some embodiments, the pharmaceutical composition is applied for about 4 days. In some embodiments, the pharmaceutical composition is applied for about 3 days.

In some embodiments, the pharmaceutical composition is applied until the wound(s) clear the skin. In some embodiments, the pharmaceutical composition is applied until the wound(s) scar.

### Combinations

Also described herein are methods for stimulating and/or enhancing wound healing in a subject in need thereof, comprising administering an additional therapeutic agent. In some embodiments, the additional therapeutic agent is an anti-inflammatory agent, an analgesic agent, an anti-bacterial agent, an anti-viral agent, or an anti-fungal agent.

In some embodiments, the additional therapeutic agent is an analgesic agent. In some embodiments, the additional therapeutic agent is benzocaine.

In some embodiments, the additional therapeutic agent is an anti-inflammatory agent. In some embodiments, the additional therapeutic agent is a steroid. In some embodiments, the additional therapeutic agent is betamethasone dipropionate, clobetasol propionate, diflorasone diacetate, fluocinonide, and halobetasol propionate, amcinonide, desoximetasone, halcinonide, triamcinolone acetonide, betamethasone valerate, clocortolone pivalate, fluocinolone acetonide, flurandrenolide, fluocinonide, fluticasone propionate, hydrocortisone butyrate, hydrocortisone valerate, mometasone furoate, prednicarbate, alclometasone dipropionate, desonide, or hydrocortisone.

In some embodiments, the additional therapeutic agent is an anti-bacterial agent. In some embodiments, the additional therapeutic agent is vancomycin, trimethoprim/sulfamethoxazole, doxycyline, ceftobiprole, ceftaroline, clindamycin, dalbavancin, daptomycin, fusidic acid, linezolid, mupirocin, omadacycline, oritavancin, tedizolid, telavancin, tigecycline, aminoglycosides, carbapenems, ceftazidime, cefepime, ceftobiprole, ceftolozane/tazobactam, fluoroquinolones, piperacillin/tazobactam, ticarcillin/clavulanic acid, linezolid, streptogramins, tigecycline, or daptomycin. in some embodiments, the additional therapeutic agent is amikacin, gentamicin, kanamycin, neomycin, netilmicin, tobramycin, paromomycin, streptomycin, spectinomycin, geldanamycin, herbimycin, rifaximin, loracarbef, ertapenem, doripenem, imipenem/cilastatin, meropenem, cefadroxil, cefazolin, cephradine, cephapirin, cephalothin, cefalexin, cefaclor, cefoxitin, cefotetan, cefamandole, cefmetazole, cefonicid, loracarbef, cefprozil, cefuroxime, cefixime, cefdinir, cefditoren, cefoperazone, cefotaxime, cefpodoxime, ceftazidime, ceftibuten, ceftizoxime, moxalactam, ceftriaxone, cefepime, ceftaroline fosamil, ceftobiprole, teicoplanin, vancomycin, telavancin, dalbavancin, oritavancin, clindamycin, lincomycin, daptomycin, azithromycin, clarithromycin, erythromycin, roxithromycin, telithromycin, spiramycin, fidaxomicin, aztreonam, furazolidone, nitrofurantoin, linezolid, posizolid, radezolid, torezolid, amoxicillin, ampicillin, azlocillin, dicloxacillin, flucloxacillin, mezlocillin, methicillin, nafcillin, oxacillin, penicillin g, penicillin v, piperacillin, penicillin g, temocillin, ticarcillin, amoxicillin/clavulanate, ampicillin/sulbactam, piperacillin/tazobactam, ticarcillin/clavulanate, bacitracin, colistin, polymyxin b, ciprofloxacin, enoxacin, gatifloxacin, gemifloxacin, levofloxacin, lomefloxacin, moxifloxacin, nadifloxacin, nalidixic acid, norfloxacin, ofloxacin, trovafloxacin, grepafloxacin, sparfloxacin, temafloxacin, mafenide, sulfacetamide, sulfadiazine, silver sulfadiazine, sulfadimethoxine, sulfamethizole, sulfamethoxazole, sulfanilimide, sulfasalazine, sulfisoxazole, trimethoprim-sulfamethoxazole (co-trimoxazole) (tmp-smx), sulfonamidochrysoidine, demeclocycline, doxycycline, metacycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, or trimethoprim. In some embodiments, the additional therapeutic agent is bacitracin, bacitracin/neomycin/polymyxin b, bacitracin/neomycin/polymyxin b/pramoxine, bacitracin/polymyxin b, erythromycin, gentamicin, mupirocin, neomycin/polymyxin b, neomycin/polymyxin b/pramoxine, ozenoxacin, retapamulin, silver sulfadiazine, sulfacetamide sodium, or sulfacetamide sodium/sulfur.

In some embodiments, the additional therapeutic agent is an anti-viral agent. in some embodiments, the additional therapeutic agent is abacavir, acyclovir, adefovir, amantadine, amprenavir, ampligen, arbidol, atazanavir, atripla, balavir, cidofovir, combivir, dolutegravir, darunavir, delavirdine, didanosine, docosanol, edoxudine, efavirenz, emtricitabine, enfuvirtide, entecavir, ecoliever, famciclovir, fomivirsen, fosamprenavir, foscarnet, fosfonet, fusion inhibitor, ibacitabine, imunovir, idoxuridine, imiquimod, indinavir, inosine, integrase inhibitor, interferon type iii, interferon type ii, interferon type i, interferon, lamivudine, lopinavir, loviride, maraviroc, moroxydine, methisazone, nelfinavir, nevirapine, nexavir, nitazoxanide, nucleoside analogues, norvir, oseltamivir, peginterferon alfa-2a, penciclovir, peramivir, pleconaril, podophyllotoxin, protease inhibitor, raltegravir, reverse transcriptase inhibitor, ribavirin, rimantadine, ritonavir, pyramidine, saquinavir, sofosbuvir, stavudine, telaprevir, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, or zidovudine.

In some embodiments, the additional therapeutic agent is an anti-fungal agent. in some embodiments, the additional therapeutic agent is amphotericin b, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, terbinafine, anidulafungin, caspofungin, or micafungin.

In some embodiments, the pharmaceutical composition described herein is administered at the same time as the additional therapeutic agent. In some embodiments, the pharmaceutical composition described herein is administered after the administration of the additional therapeutic agent. In some embodiments, the pharmaceutical composition described herein is administered before the administration of the additional therapeutic agent. In some embodiments, the pharmaceutical composition described herein is administered every time the additional therapeutic agent is administered. In some embodiments, the pharmaceutical composition described herein is administered in between administrations of the additional therapeutic agent.

In some embodiments, the additional therapeutic agent is administered once a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered twice a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered three times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered four times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered five times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered six times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered seven times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered eight times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered nine times a day and the pharmaceutical composition described herein is administered once a day, twice a day, three times a day, four times a day, five times a day, six times a day, seven times a day, eight times a day, nine times a day, or ten times a day. In some embodiments, the additional therapeutic agent is administered ten times a day and the pharmaceutical composition described herein is administered once a day, twice a day, three times a day, four times a day, five times a day, six times a day, seven times a day, eight times a day, nine times a day, or ten times a day.

In some embodiments, the pharmaceutical composition described herein is administered as a topical formulation and the additional therapeutic agent is administered as an oral formulation. In some embodiments, the pharmaceutical composition described herein is administered as a topical formulation and the additional therapeutic agent is administered as a topical formulation.

In some embodiments, the pharmaceutical composition described herein and the additional therapeutic agent are co-formulated.

### Kits/Article of Manufacture

Disclosed herein, in certain embodiments, are kits and articles of manufacture for use with one or more methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

For example, the container(s) include topical pharmaceutical composition or wound dressing described herein comprising a quaternary ammonium salt. In some embodiments, the quaternary ammonium salt is alkyl(ethylbenzyl)dimethylammonium chloride (e.g., C₁₂-C₁₄ alkyl(ethylbenzyl)dimethylammonium chloride or C₁₂ alkyl(ethylbenzyl)dimethylammonium chloride). In some embodiments, the pharmaceutical composition or wound dressing further comprises ammonium chloride. In some embodiments, the pharmaceutical composition or wound dressing further comprises a chlorite salt. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions or wound dressings are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In one embodiment, compositions containing a compound provided herein formulated in a compatible pharmaceutical carrier are also prepared, placed in an appropriate container, and labeled for treatment of an indicated condition.

### Non-Limiting Embodiments

The present disclosure is also described and demonstrated by way of the following non-limiting embodiments. However, the use of these and other embodiments anywhere in the specification is illustrative only and in no way limits the scope and meaning of the disclosure or of any exemplified term. Likewise, the disclosure is not limited to any particular preferred embodiment or aspect described herein. Indeed, suitable modifications and variations may be apparent to those skilled in the art upon reading this specification, and such variations can be made without departing from the disclosure in spirit or in scope.
1. A method for treating at least one wound in a subject in need thereof, comprising applying a therapeutically effective amount of a topical pharmaceutical composition comprising a quaternary ammonium salt.
2. A method for stimulating and/or enhancing wound healing in a subject in need thereof, comprising applying a therapeutically effective amount of a topical pharmaceutical composition comprising a quaternary ammonium salt.
3. A method for accelerating wound healing in a subject in need thereof, comprising applying a therapeutically effective amount of a topical pharmaceutical composition comprising a quaternary ammonium salt.
4. The method of any one of embodiments 1-3, wherein the quaternary ammonium salt is C₄-C₂₀ alkyl(ethylbenzyl)dimethylammonium chloride.
5. The method of any one of embodiments 1-3, wherein the quaternary ammonium salt is C₆-C₂₀ alkyl(ethylbenzyl)dimethylammonium chloride.
6. The method of any one of embodiments 1-3, wherein the quaternary ammonium salt is C₆-C₁₈ alkyl(ethylbenzyl)dimethylammonium chloride.
7. The method of any one of embodiments 1-3, wherein the quaternary ammonium salt is C₈-C₁₈ alkyl(ethylbenzyl)dimethylammonium chloride.
8. The method of any one of embodiments 1-3, wherein the quaternary ammonium salt is C₈-C₁₆ alkyl(ethylbenzyl)dimethylammonium chloride.
9. The method of any one of embodiments 1-3, wherein the quaternary ammonium salt is C₁₀-C₁₆ alkyl(ethylbenzyl)dimethylammonium chloride.
10. The method of any one of embodiments 1-3, wherein the quaternary ammonium salt is C₁₀-C₁₄ alkyl(ethylbenzyl)dimethylammonium chloride.
11. The method of any one of embodiments 1-3, wherein the quaternary ammonium salt is C₁₂-C₁₄ alkyl(ethylbenzyl)dimethylammonium chloride.
12. The method of any one of embodiments 1-3, wherein the quaternary ammonium salt is C₁₂ alkyl(ethylbenzyl)dimethylammonium chloride.
13. The method of any one of embodiments 1-3, wherein the quaternary ammonium salt is C₁₄ alkyl(ethylbenzyl)dimethylammonium chloride.
14. The method of any one of embodiments 1-13, wherein the quaternary ammonium salt is present in an amount ranging from about 0.0001% to about 40% w/w.
15. The method of any one of embodiments 1-13, wherein the quaternary ammonium salt is present in an amount ranging from about 0.0001% to about 20% w/w.
16. The method of any one of embodiments 1-13, wherein the quaternary ammonium salt is present in an amount ranging from about 0.0001% to about 10% w/w.
17. The method of any one of embodiments 1-13, wherein the quaternary ammonium salt is present in an amount ranging from about 0.0001% to about 5% w/w.
18. The method of any one of embodiments 1-13, wherein the quaternary ammonium salt is present in an amount ranging from about 0.0001% to about 4% w/w.
19. The method of any one of embodiments 1-13, wherein the quaternary ammonium salt is present in an amount ranging from about 0.0001% to about 3% w/w.
20. The method of any one of embodiments 1-13, wherein the quaternary ammonium salt is present in an amount ranging from about 0.0001% to about 2% w/w.
21. The method of any one of embodiments 1-13, wherein the quaternary ammonium salt is present in an amount ranging from about 0.0001% to about 1% w/w.
22. The method of any one of embodiments 1-13, wherein the quaternary ammonium salt is present in an amount ranging from about 0.0001% to about 0.1% w/w.
23. The method of any one of embodiments 1-13, wherein the quaternary ammonium salt is present in an amount ranging from about 0.0001% to about 0.01% w/w.
24. The method of any one of embodiments 1-13, wherein the quaternary ammonium salt is present in an amount ranging from about 0.0001% to about 0.001% w/w.
25. The method of any one of embodiments 1-13, wherein the quaternary ammonium salt is present in an amount ranging from about 0.001% to about 1% w/w.
26. The method of any one of embodiments 1-13, wherein the quaternary ammonium salt is present in an amount ranging from about 0.01% to about 1% w/w.
27. The method of any one of embodiments 1-13, wherein the quaternary ammonium salt is present in an amount ranging from about 0.1% to about 1% w/w.
28. The method of any one of embodiments 1-27, wherein the pharmaceutical composition further comprises pharmaceutically acceptable carrier.
29. The method of embodiment 28, wherein the pharmaceutically acceptable carrier comprises water, one or more organic solvents, or combinations thereof.
30. The method of any one of embodiments 28-29, wherein the pharmaceutically acceptable carrier comprises one or more gelling agents, preferably one or more gelling polymers.
31. The method of any one of embodiments 28-30, wherein the pharmaceutically acceptable carrier comprises an ointment base.
32. The method of any one of embodiments 28-31, wherein the pharmaceutically acceptable carrier comprises particles, preferably microparticles and/or nanoparticles.
33. The method of any one of embodiments 1-32, wherein the pharmaceutical composition is in the form of a solution.
34. The method of any one of embodiments 1-32, wherein the pharmaceutical composition is in the form of a gel, preferably a hydrogel.
35. The method of any one of embodiments 1-32, wherein the pharmaceutical composition is in the form of an ointment.
36. The method of any one of embodiments 1-32, wherein the pharmaceutical composition is in the form of a powder.
37. The method of any one of embodiments 1-32, wherein the pharmaceutical composition is in the form of a film.
38. The method of any one of embodiments 1-37, wherein the pharmaceutical composition further comprises at least one pharmaceutically acceptable excipient.
39. The method of embodiment 38, wherein the at least one pharmaceutically acceptable excipient comprises one or more buffering agents.
40. The method of embodiment 38-39, wherein the at least one pharmaceutically acceptable excipient comprises one or more viscosity agents.
41. The method of embodiment 38-40, wherein the at least one pharmaceutically acceptable excipient comprises one or more permeation enhancers.
42. The method of embodiment 38-41, wherein the at least one pharmaceutically acceptable excipient comprises one or more surfactants.
43. The method of embodiment 38-42, wherein the at least one pharmaceutically acceptable excipient comprises one or more stabilizers.
44. The method of embodiment 38-43, wherein the at least one pharmaceutically acceptable excipient comprises one or more emulsifiers.
45. The method of embodiment 38-44, wherein the at least one pharmaceutically acceptable excipient comprises one or more preservatives.
46. The method of embodiment 38-45, wherein the at least one pharmaceutically acceptable excipient comprises one or more thickening agents.
47. The method of any one of embodiments 1-46, wherein the pharmaceutical composition is incorporated into a wound care product.
48. The method of embodiment 47, wherein the pharmaceutical composition is incorporated into a wound care product as a coating.
49. The method of embodiment 47, wherein the pharmaceutical composition is impregnated throughout a wound care product.
50. The method of any one of embodiments 1-49, wherein the wound care product is a wound dressing.
51. The method of any one of embodiments 1-49, wherein the wound care product is a gauze.
52. The method of any one of embodiments 1-49, wherein the wound care product is a bandage.
53. The method of any one of embodiments 1-49, wherein the wound care product is a foam.
54. The method of any one of embodiments 1-49, wherein the wound care product is a cohesive wrap.
55. The method of any one of embodiments 1-49, wherein the wound care product is a wound pad, plaster or absorbent lint.
56. The method of any one of embodiments 1-49, wherein the wound care product is a wound wash, and optionally wherein the wound wash is a surgical irrigation liquid or operative debridement liquid.
57. The method of any one of embodiments 1-49, wherein the wound care product is a surgical wash or a surgical scrub.
58. The method of any one of embodiments 1-57, wherein the wound is an open wound.
59. The method of any one of embodiments 1-57, wherein the wound is an incision, an abrasion, a laceration, a puncture, or an avulsion.
60. The method of any one of embodiments 1-57, wherein the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof.
61. The method of any one of embodiments 1-57, wherein the wound is a burn.
62. The method of any one of embodiments 1-61, wherein the wound is infected.
63. The method of embodiment 62, wherein the infected wound is infected with methicillin-resistant Staphylococcus aureus (MRSA).
64. The method of any one of embodiments 1-63, wherein the subject is a human.
65. The method of any one of embodiments 1-64, wherein the subject is immuno-compromised.
66. A wound care product comprising a pharmaceutical composition, wherein the pharmaceutical composition comprises a quaternary ammonium salt.
67. The wound care product of embodiment 66, wherein the quaternary ammonium salt is C₄-C₂₀ alkyl(ethylbenzyl)dimethylammonium chloride.
68. The wound care product of embodiment 66, wherein the quaternary ammonium salt is C₆-C₂₀ alkyl(ethylbenzyl)dimethylammonium chloride.
69. The wound care product of embodiment 66, wherein the quaternary ammonium salt is C₆-C₁₈ alkyl(ethylbenzyl)dimethylammonium chloride.

70. The wound care product of embodiment 66, wherein the quaternary ammonium salt is C₈-C₁₈ alkyl(ethylbenzyl)dimethylammonium chloride.
71. The wound care product of embodiment 66, wherein the quaternary ammonium salt is C₈-C₁₆ alkyl(ethylbenzyl)dimethylammonium chloride.
72. The wound care product of embodiment 66, wherein the quaternary ammonium salt is C₁₀-C₁₆ alkyl(ethylbenzyl)dimethylammonium chloride.
73. The wound care product of embodiment 66, wherein the quaternary ammonium salt is C₁₀-C₁₄ alkyl(ethylbenzyl)dimethylammonium chloride.
74. The wound care product of embodiment 66, wherein the quaternary ammonium salt is C₁₂-C₁₄ alkyl(ethylbenzyl)dimethylammonium chloride.
75. The wound care product of embodiment 66, wherein the quaternary ammonium salt is C₁₂ alkyl(ethylbenzyl)dimethylammonium chloride.
76. The wound care product of embodiment 66, wherein the quaternary ammonium salt is C₁₄ alkyl(ethylbenzyl)dimethylammonium chloride.
77. The wound care product of any one of embodiments 66-76, wherein the quaternary ammonium salt is present in an amount ranging from about 0.0001% to about 40% w/w.
78. The wound care product of any one of embodiments 66-76, wherein the quaternary ammonium salt is present in an amount ranging from about 0.0001% to about 20% w/w.
79. The wound care product of any one of embodiments 66-76, wherein the quaternary ammonium salt is present in an amount ranging from about 0.0001% to about 10% w/w.
80. The wound care product of any one of embodiments 66-76, wherein the quaternary ammonium salt is present in an amount ranging from about 0.0001% to about 5% w/w.
81. The wound care product of any one of embodiments 66-76, wherein the quaternary ammonium salt is present in an amount ranging from about 0.0001% to about 4% w/w.
82. The wound care product of any one of embodiments 66-76, wherein the quaternary ammonium salt is present in an amount ranging from about 0.0001% to about 3% w/w.
83. The wound care product of any one of embodiments 66-76, wherein the quaternary ammonium salt is present in an amount ranging from about 0.0001% to about 2% w/w.
84. The wound care product of any one of embodiments 66-76, wherein the quaternary ammonium salt is present in an amount ranging from about 0.0001% to about 1% w/w.
85. The wound care product of any one of embodiments 66-76, wherein the quaternary ammonium salt is present in an amount ranging from about 0.0001% to about 0.1% w/w.
86. The wound care product of any one of embodiments 66-76, wherein the quaternary ammonium salt is present in an amount ranging from about 0.0001% to about 0.01% w/w.
87. The wound care product of any one of embodiments 66-76, wherein the quaternary ammonium salt is present in an amount ranging from about 0.0001% to about 0.001% w/w.
88. The wound care product of any one of embodiments 66-76, wherein the quaternary ammonium salt is present in an amount ranging from about 0.001% to about 1% w/w.
89. The wound care product of any one of embodiments 66-76, wherein the quaternary ammonium salt is present in an amount ranging from about 0.01% to about 1% w/w.
90. The wound care product of any one of embodiments 66-76, wherein the quaternary ammonium salt is present in an amount ranging from about 0.1% to about 1% w/w.
91. The wound care product of any one of embodiments 66-90, wherein the pharmaceutical composition further comprises pharmaceutically acceptable carrier.
92. The wound care product of embodiment 91, wherein the pharmaceutically acceptable carrier comprises water, one or more organic solvents, or combinations thereof.
93. The wound care product of any one of embodiments 91-92, wherein the pharmaceutically acceptable carrier comprises one or more gelling agents, preferably one or more gelling polymers.
94. The wound care product of any one of embodiments 91-93, wherein the pharmaceutically acceptable carrier comprises an ointment base.
95. The wound care product of any one of embodiments 91-94, wherein the pharmaceutically acceptable carrier comprises particles, preferably microparticles and/or nanoparticles.
96. The wound care product of any one of embodiments 66-95, wherein the pharmaceutical composition is in the form of a solution, preferably a wound wash.
97. The wound care product of any one of embodiments 66-95, wherein the pharmaceutical composition is in the form of a gel, preferably a hydrogel.
98. The wound care product of any one of embodiments 66-95, wherein the pharmaceutical composition is in the form of an ointment.
99. The wound care product of any one of embodiments 66-95, wherein the pharmaceutical composition is in the form of a powder.
100. The wound care product of any one of embodiments 66-95, wherein the pharmaceutical composition is in the form of a film.
101. The wound care product of any one of embodiments 66-100, wherein the pharmaceutical composition further comprises at least one pharmaceutically acceptable excipient.
102. The wound care product of embodiment 101, wherein the at least one pharmaceutically acceptable excipient comprises one or more buffering agents.
103. The wound care product of embodiment 101, wherein the at least one pharmaceutically acceptable excipient comprises one or more viscosity agents.
104. The wound care product of embodiment 101, wherein the at least one pharmaceutically acceptable excipient comprises one or more permeation enhancers.
105. The wound care product of embodiment 101, wherein the at least one pharmaceutically acceptable excipient comprises one or more surfactants.
106. The wound care product of embodiment 101, wherein the at least one pharmaceutically acceptable excipient comprises one or more stabilizers.
107. The wound care product of embodiment 101, wherein the at least one pharmaceutically acceptable excipient comprises one or more emulsifiers.
108. The wound care product of embodiment 101, wherein the at least one pharmaceutically acceptable excipient comprises one or more preservatives.
109. The wound care product of embodiment 101, wherein the at least one pharmaceutically acceptable excipient comprises one or more thickening agents.
110. The wound care product of any one of embodiments 66-109, wherein the pharmaceutical composition is incorporated into the wound care product as a coating.
111. The wound care product of any one of embodiments 66-109, wherein the pharmaceutical composition is impregnated throughout the wound care product.
112. The wound care product of any one of embodiments 66-111, wherein the wound care product is a wound wash, and optionally wherein the wound wash is a surgical irrigation liquid or operative debridement liquid.
113. The wound care product of any one of embodiments 66-111, wherein the wound care product is a surgical wash or surgical scrub.
114. The wound care product of any one of embodiments 66-111, wherein the wound care product is a wound dressing.
115. The wound care product of any one of embodiments 66-111, wherein the wound care product is a gauze.
116. The wound care product of any one of embodiments 66-111, wherein the wound care product is a bandage.
117. The wound care product of any one of embodiments 66-111, wherein the wound care product is a foam.
118. The wound care product of any one of embodiments 66-111, wherein the wound care product is a cohesive wrap.
119. The wound care product of any one of embodiments 66-111, wherein the wound care product is a wound pad.
120. The wound care product of any one of embodiments 66-111, wherein the wound care product is a wound plaster.
121. The wound care product of any one of embodiments 66-111, wherein the wound care product is an absorbent lint.

### NON-LIMITING EXAMPLES

The present disclosure is also described and demonstrated by way of the following non-limiting examples. However, the use of these and other examples anywhere in the specification is illustrative only and in no way limits the scope and meaning of the disclosure or of any exemplified term. Likewise, the disclosure is not limited to any particular embodiment or aspect described herein. Indeed, suitable modifications and variations may be apparent to those skilled in the art upon reading this specification, and such variations can be made without departing from the disclosure in spirit or in scope.

Formulations A and B are as follow:

| | Formulation A | Formulation B |
|---|---|---|
| Ingredient | % (w/w) | % (w/w) |
| sodium phosphate, heptahydrate, | 0.195 | 0.195 |
| dibasic | | |
| sodium phosphate, monohydrate, | 0.012 | 0.012 |
| monobasic | | |
| sodium chloride | 0.10 | 0.10 |
| potassium chloride | 0.20 | 0.20 |
| Tetronic 908 | 0.250 | 0.250 |
| ammonium chloride | 0.25 | 0.25 |
| Stabilized chlorine dioxide (as a | 0.02 | 0.040 |
| sodium chlorite solution) | | |
| C12-C14- | 0.0150 | 0.0200 |
| alkyl(ethylbenzyl)dimethylammonium | | |
| chloride | | |
| propylene glycol | 0.75 | 0.75 |
| EDTA | 0.05 | 0.05 |

### Example 1: Murine Model of Wound Healing

An exemplary formulation (Formulation A) was tested in the murine model of wound healing (Dunn, Louise, et al. “Murine model of wound healing.” Journal of Visualized Experiments. 75 (2013): e50265-e50265).

### On Day 0:

Biopsy punch model performed
Infected with 1×10⁵ CFUs bioluminescent MRSA

Wound was imaged then treated with Formulation A

### Days 1-2

The animals were treated topically with 20 μL of Formulation A, bacitracin, or vehicle three times daily and imaged

### Days 3-14

The animals were imaged, weight, and their wound was measured daily

### Day 14

The animals were sacrificed and the bacterial burdens were checked in the wound, the liver, the spleen, and the blood.

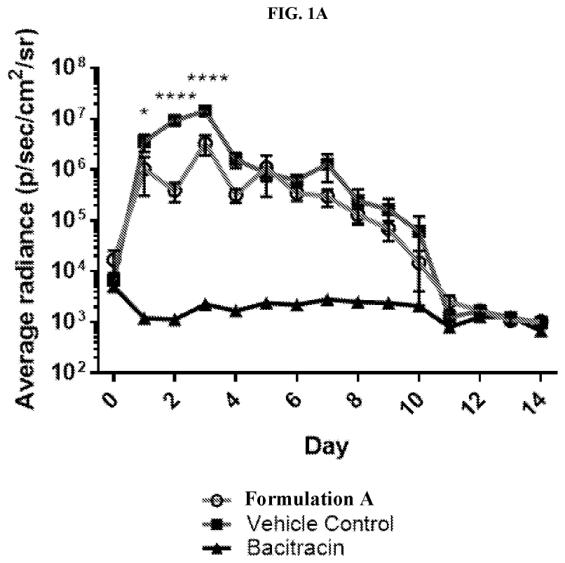

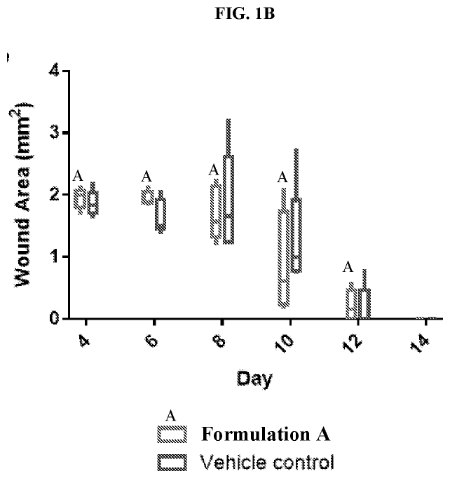

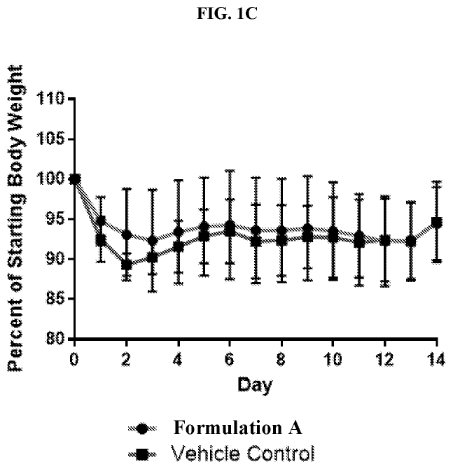

Formulation A Inhibits the Growth of CA-MRSA in the Biopsy Wound Model as can be seen in FIG. 1A, FIG. 1B, and FIG. 1C. Tracking of infection and wound healing in mice infected with 1×10⁵ CFU bioluminescent MRSA and treated with 20 μL Formulation A, bacitracin (positive control), or vehicle control (N=5 for each treatment group). FIG. 1A: average radiance in the wound bed detected over a two week period. Bacitracin treated mice completely cleared infection within the first day as expected. Mice treated with Formulation A had significantly reduced burdens on days 1, 2, and 3. Statistical analysis was conducted in GraphPad Prism 6 using a two-way ANOVA test: ***p<0.001; ****p<0.0001. FIG. 1B: box and whisker plots of the wound area over time, beginning on day 4 after the removal of the surgical wound dressing. There is no significant difference in the rate of wound closure between the two treatment groups, and by the day 14 end point, the wounds had completely closed for all of the treatment groups. FIG. 1C: plot of the change in weight through the duration of infection and treatment. There was no significant difference in weight change between the treatment groups, indicating that treatment with the Formulation A biocide is not having a detectable detrimental effect on body weight.

### Formula A Limits Biofilm Development on Surgical Dressing

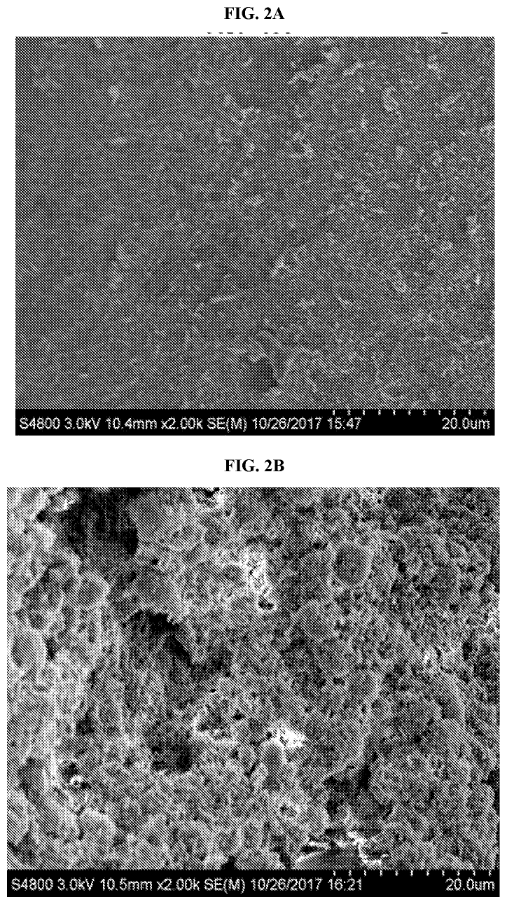

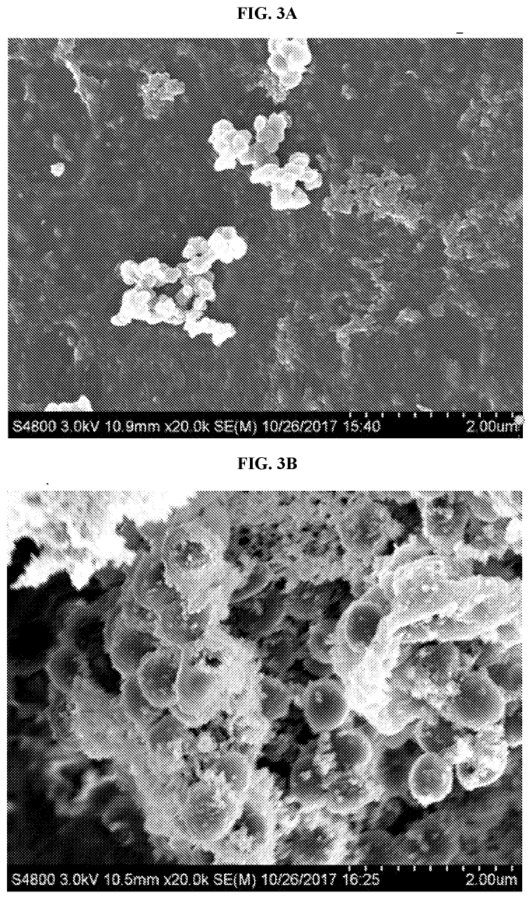

On day three of infection, the wound dressing was removed from each mouse. From each group of mice, 3 were chosen at random to have their wound dressing fixed and analyzed with a scanning electron microscope to assess biofilm formation. For each sample, 5 images were taken at a magnification of 2,000× (FIG. 2A: Formulation A and FIG. 2B: vehicle control) and 20,000× (FIG. 3A: Formulation A and FIG. 3B: vehicle control) and representative images were selected. The wound dressing from the Formulation A treated mice appears to primarily consist of cellular debris (FIG. 2A and FIG. 3A). In contrast, the wound dressing from the control mice was covered in a dense biofilm with discernable staphylococci at the 20,000× magnification (FIG. 2B and FIG. 3B).

### Formula A Limits CA-MRSA Growth in the Wound

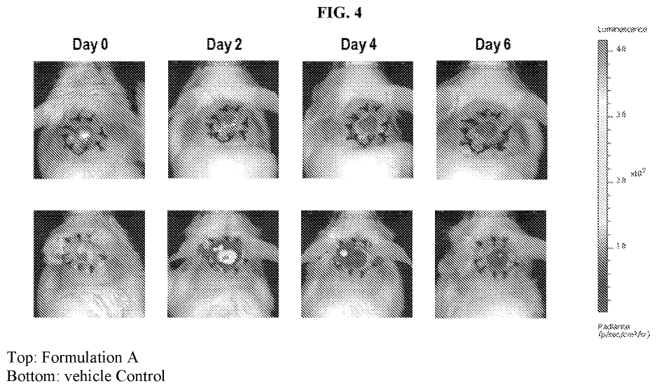

Representative images of a single mouse from each treatment group over the course of the first week of infection in the infection and treatment model shown in FIG. 4. Mice were infected with 1×10⁵ CFUs of bioluminescent CA-MRSA and imaged daily. In the vehicle control mouse (bottom), we see infection spread throughout the wound bed completely uninhibited and even extend to skin beyond the silicone ring, while the treated mouse (top) has no detectable bioluminescence until the end of the first week.

### Example 2: Full Thickness Splinted Punch Wound Model

On day 0, mice were wounded and the wound bed directly infected with SAP227. Animals were anesthetized by an intraperitoneal (IP) injection of ketamine (90 mg/kg) and xylazine (10 mg/kg) mixture. A separate IP injection of buprenorphine (0.05 mg/kg) was also administered to prophylactically accommodate for pain. Once fully anesthetized, the dorsum was shaved and the exposed skin was scrubbed with chlorhexidine to sanitize the surface. Thereafter, a full-thickness wound was generated using a 5 mm biopsy punch (Integra Miltex) by application of pressure on the dorsum below the base of the skull and between the solar plexus, generating a 5 mm circular wound outline. The light perforation was then excised and the skin was cut through the epidermis, dermis, and panniculus carnosis exposing the muscle beneath. Thereafter, a 10 mm silicone torus coated in surgical adhesive was placed over the wound. Tegaderm (3M Healthcare) was placed over the silicone torus covering the exposed wound bed and the torus was secured in place using 4-0 braided silk interrupted sutures (Ethicon) fortified with additional surgical adhesive. A 138 n insulin syringe was used to experimentally infect the wound by penetrating the tegaderm and expressing directly onto the wound bed. Mice were monitored daily to access bacterial burdens, wound closure, and weight change for the duration of the study until sacrifice on day 14. Daily injections of buprenorphine (0.05 mg/kg) were performed for the initial 48 hours post-innoculation to accommodate for any postsurgical pain.

Treatment. Animals were allowed to recover for four hours post wounding surgery and inoculation and then anesthetized via inhalation (2.5%, isoflurane). Thereafter either Formulation B, Bacitracin, or vehicle control was administered via injection through the tegaderm covering to separate groups (n=5). Treatments were provided every eight hours for the first three days of infection (total of 9 treatments).

Observations and clinical scoring: Animals were monitored and weighed daily to day +14 to assess health in response to infection and treatment. Individual wounds were examined daily, clinical status assessed, and categorically scored using the following criteria: 0=no redness, swelling, or discharge; 1=light inflammation and some discharge; 2=redness, swelling, discharge, and/or discoloration; 3=heavy inflammation, discoloration, and purulence.

Quantification of bacterial burdens and wound closure: To determine bacterial burdens within the wound over time and examine the rate at which the wound was healing, mice were imaged daily using an in vivo imaging system (IVIS)-XMRS (PerkinElmer). The IVIS is capable of detecting and quantifying bioluminescent signals used to track the infection progression of the bioluminescent bacteria within the wound. During use, mice were anesthetized via inhalation of 2.5% isoflurane and imaged individually for 60 s. Resulting images were analyzed using the IVIS Lumina Living Image Software (PerkinElmer). A circular region of interest (RoI) was electronically captured over each wound bed to quantify bioluminescence and bacterial burdens, which was used for every mouse every day. Relative luminescence within t 162 he RoI in units of radiance (photons/centemeter2/steradian/second) is interpolated by companion system software. Animals were humanely euthanized on day 14 via CO2 asphyxiation, and tissues sampled to further quantify bacterial burdens by excising the wound, which extended roughly 5 mm beyond the wound edge. Tissue samples were mechanically disrupted in 1 mL sterile 1×PBS, serially diluted, and plated on LB agar. Resulting bacterial colonies were imaged (IVIS) to verify the presence of CA-MRSA. In addition, the spleen and liver were removed and processed in a similar manner for determination of whether the bacteria had disseminated from the wound. The rate and progression of wound healing was monitored for animals using custom RoIs traced along the epithelial lip of each wound (IVIS) that generated a wound area estimate. Wound area measurements were performed daily beginning upon the removal of the tegaderm covering, and provided a quantitative assessment of the rate and progression of wound healing over time.

### Formulation B is Bactericidal to Clinically-Relevant MRSA Strains In Vitro and In Vivo.

The MIC and MBC of Formulation B was initially determined using a panel of S. aureus strains (Table 1) including laboratory S. aureus ATCC 6538, bioluminescent S. aureus strain Newman, bioluminescent CA-MRSA strain MW2, and a MRSA clinical isolate.

TABLE 1

Minimum Inhibitory Concentration

| | | | Bacitracin |
|---|---|---|---|
| S. aureus strain | Treatment | Control | (μg/mL) |
| S. aureus ATCC 6538 | 1:125 | >1:5 | 67 |
| MRSA clinical isolate | 1:25 | >1:5 | 67 |
| Bioluminescent CA-MRSA | 1:125 | >1:5 | 67 |
| strain MW2 | | | |
| Bioluminescent S. aureus | 1:125 | >1:5 | 67 |
| strain Newman | | | |

Minimum Bactericidal Concentration

| | | | Bacitracin |
|---|---|---|---|
| S. aureus strain | Treatment | Control | (μg/mL) |
| S. aureus ATCC 6538 | 1:25 | >1:5 | 67 |
| MRSA clinical isolate | 1:25 | >1:5 | 67 |
| Bioluminescent CA-MRSA | 1:25 | >1:5 | 333 |
| strain MW2 | | | |
| Bioluminescent S. aureus | 1:5 | >1:5 | 333 |
| strain Newman | | | |

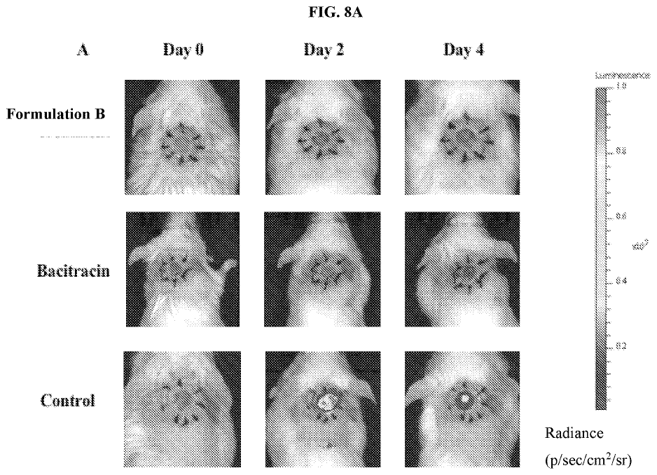

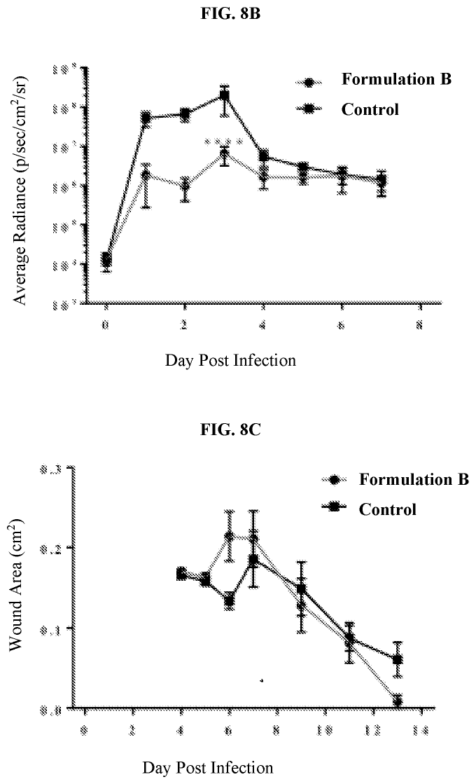

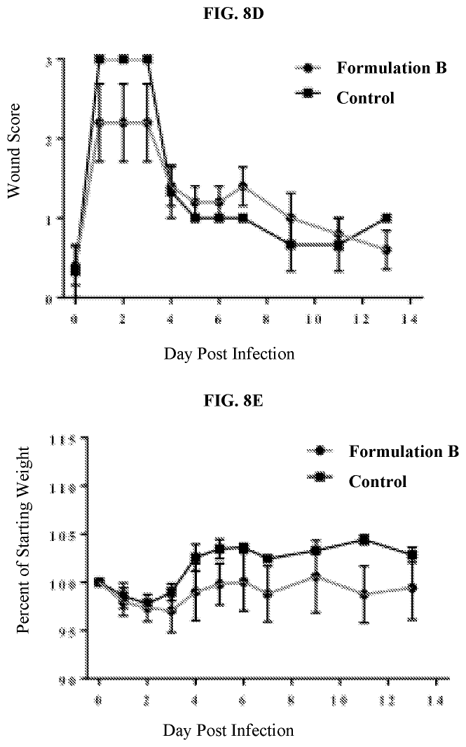

Formulation B demonstrated potent in vitro inhibitory activity against all four strains of S. aureus tested, and inhibited CA-MRSA (1:125 dilution) and the MRSA clinical isolate (1:25 dilution). The resulting MICs for bacitracin against every MRSA strain tested (67 μg/mL) approximated the dilution (and subsequent dose) used in the murine wound treatment experiments, which equated to the inhibitory capacity of bacitracin against MRSA in vivo. Similarly, Formulation B was bactericidal at a 1:25 dilution in MBC determination for three of the four strains tested, which roughly equated to the dilution factor for bacitracin against CA-MRSA. Significant inhibition of CA-MRSA was observed in the wound bed over the three days of treatment in Formulation B-treated mice (FIG. 8A).

Nearly all (⅘; 80%) of Formulation B-treated mice had a 250 significant reduction of bacterial burdens through the duration of the three-day treatment period (FIG. 8B), however some (⅗; 60%) showed slight increases in bacterial luminescence upon the cessation of treatment. Vehicle control-treated animals demonstrated unfettered bacterial growth similar to the pattern of what was observed when establishing the CA-MRSA infection wound model. The gross clinical pathology categorical scoring showed vehicle control-treated mice experiencing purulent, clearly infected wounds +2d to +3d post infection procedure. In contrast, 3 of five (⅗; 60%) Formulation B-treated animals developed any gross clinical changes associated with infection (FIG. 8D). Wound closure and weight change were not significantly different among experimental groups (FIG. 8C and FIG. 8E), suggesting that treatment with Formulation B does not deter wound closure or negatively impact the mice.

### Example 3: S. mutans Quantitative Biofilm Reduction Assay

### Protocol:

Day 1: O/N culture of S. mutans
Day 2: Dilute O/N culture 1:9 in BHI
   Place discs in 2 mL diluted culture and incubate 24 h
Day 3 Transfer discs to 2 mL BMGS (culture in BMGS for 1-3 days)
Day 4 1-Day: Transfer discs to 2 mL sterilants for desired incubation time
   Transfer discs back to 2 mL BMGS for 24 h
2-Day: Transfer discs to 2 mL BMGS and incubate for another 24 h
Day 5 1-Day: Sonicate disc and plate 100 μL of different dilutions
   2-Day: Transfer discs to 2 mL sterilants for desired incubation time
      Transfer discs back to 2 mL BMGS for 24 h
Day 6 1-Day: Count plates
   2-Day: Sonicate disc and plate 100 μL of different dilutions
Day 7 Count plates

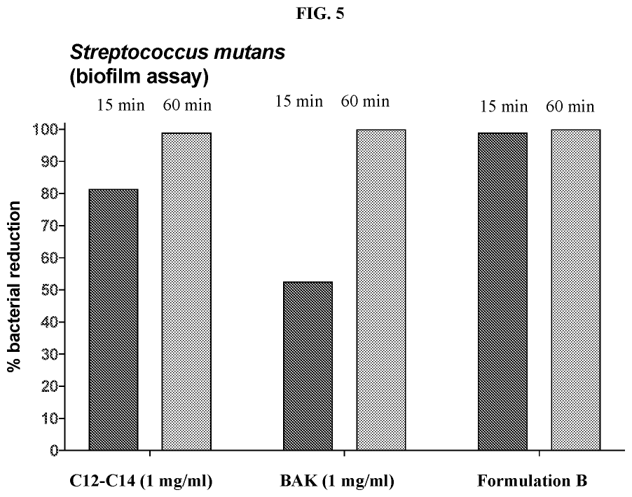

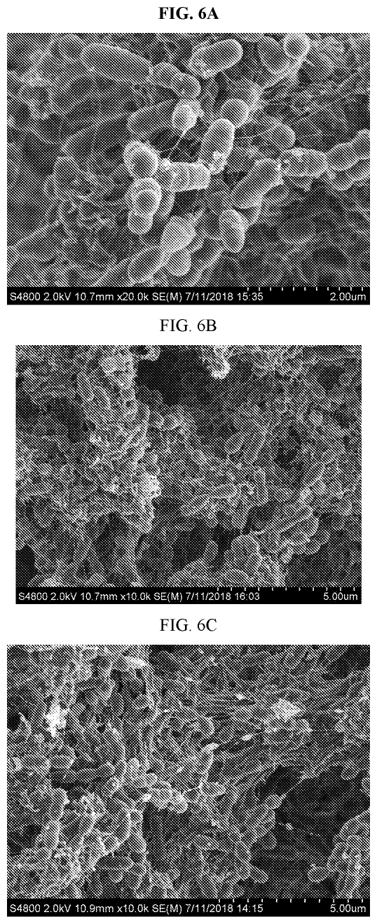

Formulation B, benzalkonium chloride (BAK) and C₁₂-C₁₄ alkyl(ethylbenzyl)dimethylammonium chloride were tested in the S. mutans quantitative biofilm reduction assay. Results are shown in FIG. 5. Formulation B is more potent than either BAK or C₁₂-C₁₄ alkyl(ethylbenzyl)dimethylammonium chloride alone at 15 minutes contact time; equivalent at 60 minutes contact time. Formulation B (FIG. 6A) showed less biofilm accumulation. C₁₂-C₁₄ alkyl(ethylbenzyl)dimethylammonium chloride (FIG. 6B) and BAK (FIG. 6C) showed approximate equivalence with biofilm formation at same treatment concentration.

### Example 4: Minimal Biofilm Eradication Concentration Protocol

### Protocol:

Day 1 Grow S. mutans overnight in BHI (5 mL+1 loop S. mutans)
Day 2 Dilute overnight culture in BMGS (1 mL S. mutans+9 mL BMGS)
   Add 80 μL diluted culture in each well of a 96-well plate
   Incubate 24 hours
Day 3 Take BMGS supernatant out
   Add 100 μL treatments (serial diluted 1:2) into each well
   Incubate 24 hours
Day 4 Wash wells once with 150 μL PBS (centrifuge @3750 rpm for 5 min, remove supernatant, add 150 μL PBS, shake, centrifuge @3750 rpm for 5 min, remove PBS)
   Add 90 μL BHI in each well
   Incubate 24 hours
Day 5 Add 190 uL fresh BHI into wells of a new 96-well plate
   Add 10 uL from each treated well into this new plate
   Read OD600 at 0 h
Day 6 Read OD600 at 24 h

### Example 5: Glass Slide Spread Plate Assay

### Solutions and Materials

S. mutans (UA159)
BHI medium (for Sm O/N culture, 5 ml)
BMGS (100 ml)
PBS (for control and counting CFU)
50 ml falcon tubes
Glass cover slide (Autoclave before use, Fisherband 12-545-M)
Spreaders (Fisherband 14-665-230)
BHI plates (To start with, 8 for each treatment)

### Growing a Biofilm (2 Days)

Grow a culture of Sm overnight in BHI medium (1 colony in 5 ml BHI). Incubate statically at 37° C., 5% CO₂.
Dilute the overnight Sm culture 1:100 into fresh BMGS. Put in glass slide, culture for 24 hrs followed with changing tubes with fresh BMGS every day for 3 days. Incubate statically at 37° C., 5% CO₂.

### Counting CFU (Time Point+2 Days)

Use a sterile tweezer to take the slide out of the tube. Use a scraper to scrape the cells into 10 ml PBS.
Disperse 1 ml of biofilm cell suspension into each EP tube. Pellet the cells at 6000 rpm for 10 min. Each tube was resuspended with 300 μl treatment.
70 μl from the tubes was taken at each time point (0 h, 3 h, 6 h and 24 h) and added in 630 μl PBS. Sonicate (lowest power for 20 sec) before serial dilutions.
100 μl from previous EP tube+900 μl PBS for making series dilutions
Take 100 μl from each dilution and plate. Incubate plates at 37° C., 5% CO₂.
Count CFUs two days later

### Example 6: Biofilm Medium (BM)

Working BM requires 1) base medium, 2) MgSO₄, 3) carbohydrate source (glucose or sucrose or both or others, final concentration around 20 mM), 4) vitamins, and 5) amino acids. Modified BM is without iron, calcium, and manganese.

| 1) Base medium (per liter) | | |
|---|---|---|
| K₂HPO₄ | 10 | g |
| KH₂PO₄ | 2 | g |
| (NH₄) ₂SO₄ | 1.3 | g |
| NaCl | 2 | g |
| FeCl₃•6H₂O | 0.001 g | |
| | (just a touch) | |
| Casamino Acids | 2.0 | g |

pH to 7.4 & Autoclave
2) Make 50 mL of 10% MgSO₄•7H₂O. Sterile filter.
3) Make 250 mL 1M Glucose stock. Sterile filter.
Good for 1 year.
Make 250 mL 1M Sucrose stock. Sterile filter.
Good for 1 year.
4) 100X amino acids stock, per 10 mL

| | | |
|---|---|---|
| L-glutamate (sodium salt) | 0.5 | g |
| L-arginine-HCl | 0.2 | g |
| L-cysteine-HCl | 0.2 | g |

| | |
|---|---|
| L-tryptophan | 0.02 g (20 mg) |

*Dissolve in 10 mL sterile water and filter sterilize.
Make fresh amino acids 3 days.
5) 100X vitamins stock, per 100 mL

| | | |
|---|---|---|
| Pyridoxine-HCl | 240 | mg |
| Nicotinic acid | 46 | mg |
| Pantothenic acid | 24 | mg |
| Riboflavin | 4 | mg |
| Thiamine-HCl | 1 | mg |
| D-Biotin | 0.12 | mg |

*Dissolve in 100 mL sterile water and filter sterilize.
Make fresh vitamins every 6 months.
Recipe for working BM medium (per 100 mL)

| Base Medium | 95 | mL |
|---|---|---|
| 10% Mg | 0.5 | mL |
| 1M Glucose | 1.8 mL and/or* | |
| 1M Sucrose | 0.2 | mL |
| 100X Vitamins | 1 | mL |
| 100X Amino Acids | 1 | mL |

*if making BMGS follow above protocol (final [glucose = 18 mM] & final [sucrose = 2 mM]); if making BMG, add 2 mL 1M glucose only (final [glucose = 20 mM]); if making BMS, add 1 mL sucrose only (final [sucrose = 10 mM]).

### Example 7: Scanning Electron Microscopy of Infected Wounds (MRSA) Treated with Formulation B

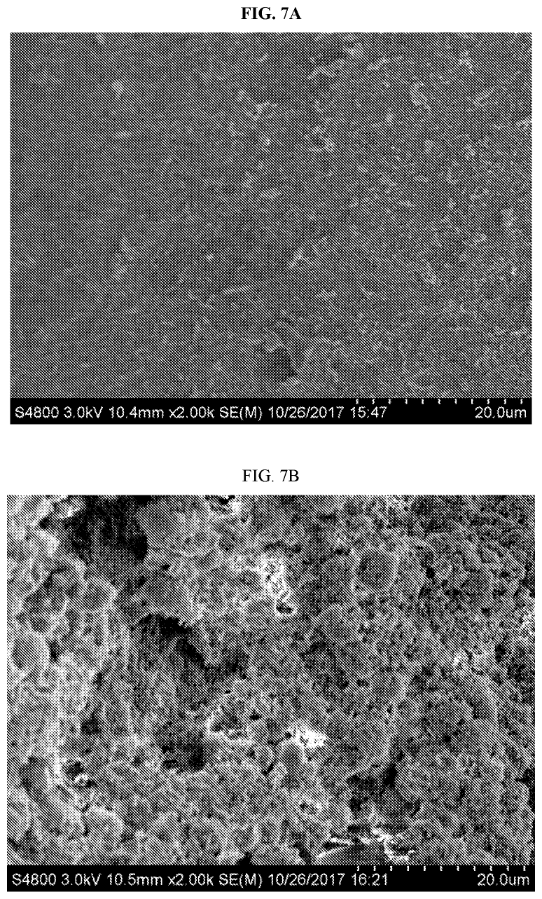

Representative SEM images of tegaderm covering the wounds of treated and control mice (each image is taken from a different mouse) magnified at 2,000×. The tegaderm from the treatment mouse (FIG. 7A) appears to have minimal bacteria is seems to mostly consist of cellular debris. The control treated mice (FIG. 7B) demonstrate a dense biofilm.

What is claimed is:

1. A method for treating at least one wound in a subject in need thereof, comprising applying a therapeutically effective amount of a topical pharmaceutical composition comprising a quaternary ammonium salt.

2. The method of claim 1, wherein the application of the topical pharmaceutical composition provides stimulating and/or enhancing wound healing in the subject in need thereof.

3. The method of claim 1, wherein the application of the topical pharmaceutical composition provides accelerating wound healing in the subject in need thereof.

4. The method of claim 1, wherein the quaternary ammonium salt is C6-C20 alkyl (ethylbenzyl)dimethylammonium chloride.

5. The method of claim 1, wherein the quaternary ammonium salt is C10-C16 alkyl (ethylbenzyl)dimethylammonium chloride.

6. The method of claim 1, wherein the quaternary ammonium salt is C12-C14 alkyl (ethylbenzyl)dimethylammonium chloride.

7. The method of claim 1, wherein the quaternary ammonium salt is C12 alkyl (ethylbenzyl)dimethylammonium chloride.

8. The method of claim 1, wherein the quaternary ammonium salt is present in an amount ranging from about 0.0001% to about 40% w/w.

9. The method of claim 1, wherein the quaternary ammonium salt is present in an amount ranging from about 0.0001% to about 10% w/w.

10. The method of claim 1, wherein the quaternary ammonium salt is present in an amount ranging from about 0.0001% to about 5% w/w.

11. The method of claim 1, wherein the quaternary ammonium salt is present in an amount ranging from about 0.0001% to about 1% w/w.

12. The method of claim 1, wherein the quaternary ammonium salt is present in an amount ranging from about 0.0001% to about 0.1% w/w.

13. The method of claim 1, wherein the quaternary ammonium salt is present in an amount ranging from about 0.0001% to about 0.01% w/w.

14. The method of claim 1, wherein the pharmaceutical composition further comprises pharmaceutically acceptable carrier.

15. The method of claim 14, wherein the pharmaceutically acceptable carrier comprises one or more gelling agents, preferably one or more gelling polymers.

16. The method of claim 14, wherein the pharmaceutically acceptable carrier comprises an ointment base.

17. The method of claim 14, wherein the pharmaceutically acceptable carrier comprises particles, preferably microparticles and/or nanoparticles.

18. The method of claim 1, wherein the pharmaceutical composition is in the form of a solution.

19. The method of claim 1, wherein the pharmaceutical composition is in the form of a gel, preferably a hydrogel.

20. The method of claim 1, wherein the pharmaceutical composition is in the form of an ointment.

21. The method of claim 1, wherein the pharmaceutical composition is in the form of a powder.

22. The method of claim 1, wherein the pharmaceutical composition is in the form of a film.

23. The method of claim 1, wherein the pharmaceutical composition is incorporated into a wound care product.

24. The method of claim 1, wherein the pharmaceutical composition is coated on or impregnated throughout the wound care product.

25. The method of claim 23, wherein the wound care product is selected from the group consisting of a wound dressing, a gauze, a bandage, a foam, and a cohesive wrap.

26. The method of claim 23, wherein the wound care product is a wound pad, plaster or absorbent lint.

27. The method of claim 23, wherein the wound care product is a wound wash, and optionally wherein the wound wash is a surgical irrigation liquid or operative debridement liquid.

28. The method of claim 23, wherein the wound care product is a surgical wash or a surgical scrub.

29. The method of claim 1, wherein the wound is an ulcer, burn, trauma wound, subcutaneous trauma wound, dermatitis, sports injury, muscle soreness, joint soreness, muscle stiffness, joint stiffness, laceration, scarring, surgical wound, arthritis, foot calluses, dry skin, back pain, bruise, or inflammation, or any combination thereof.

30. The method of claim 1, wherein the wound is an infected wound.
